(19) United States
(12) Patent Application Publication
(10) Pub. No.: US 2024/0392101 A1
(43) Pub. Date: Nov. 28, 2024

(54) PLASMONIC VIS-NIR PHOTOTHERMAL ACTIVATION OF OLEFIN METATHESIS ENABLING PHOTORESPONSIVE MATERIALS

(71) Applicant: B. G. NEGEV TECHNOLOGIES AND APPLICATIONS LTD., Beer Sheva (IL)

(72) Inventors: Yossef WEIZMANN, Beer Sheva (IL); Nir LEMCOFF, Beer Sheva (IL)

(73) Assignee: B. G. NEGEV TECHNOLOGIES AND APPLICATIONS LTD., Beer Sheva (IL)

(21) Appl. No.: 18/790,173

(22) Filed: Jul. 31, 2024

Related U.S. Application Data
(63) Continuation of application No. PCT/IL2023/050092, filed on Jan. 26, 2023.
(60) Provisional application No. 63/305,692, filed on Feb. 2, 2022.
(60) Provisional application No. 63/356,495, filed on Jun. 29, 2022.

(51) Int. Cl. C08K 3/105; B01J 23/52; B01J 31/16; B01J 31/18; B01J 31/22; C08G 61/08; C08K 3/36; C08K 9/02

(52) U.S. Cl. CPC C08K 3/105; B01J 23/52; B01J 31/1691; B01J 31/185; B01J 31/226; C08G 61/08; C08K 3/36; C08K 9/02; C08G 2261/11; C08G 2261/3325; C08G 2261/418; C08K 2201/005

(57) ABSTRACT

[symbol]

plasmonic ROM polymer composites optionally comprising a latent catalyst, precursors thereof and uses thereof as a catalyst, are disclosed.

### CROSS-REFERENCE TO RELATED APPLICATIONS

This application is a Bypass Continuation of PCT Patent Application No. PCT/IL2023/050092 having International filing date of Jan. 26, 2023, which claims the benefit of priority of U.S. Provisional Patent Application Nos. 63/305,692 filed Feb. 2, 2022, and 63/356,495 filed Jun. 29, 2022 both titled “Plasmonic Vis-N5R Photothermal Activation Of Elefin Metathesis Enabling Photoresponsive Materials”. The contents of these applications are all incorporated herein by reference in their entirety.

### FIELD OF THE INVENTION

The invention relates generally to the field of plasmonic polymeric composites and articles comprising same.

### BACKGROUND OF THE INVENTION

The use of plasmonic nanoparticles as light-to-heat converters via the localized surface plasmon resonance (LSPR) effect has been steadily expanding since its application as an optical label for large molecules almost two decades ago. It has since been utilized in various fields, including biosensing, photocatalysis, and most notably in therapeutics for photothermal cancer therapy. Gold nanoparticles stand out in photothermal applications due to their exceptional plasmonic properties, biocompatibility, and chemical stability. Moreover, the fine control over shape and size allows the development of diverse structures from simple spheres to more complex anisotropic geometries such as nanostars, rods, and bipyramids. The motivation for developing new structures stems from the LSPR being dependent on the exact nanoparticle geometry, therefore, controlling the structure of nanoparticles translates directly to controlling the properties of light-to-heat conversion. Gold bipyramids (AuBPs) are especially suited as nanoconverters due to a well-defined control over size and monodispersity, resulting in tunable narrow LSPR bands and remarkable heating efficiency, compared to other nanostructures. Integrating plasmonic nanoparticles into organic synthesis to exploit the photothermal effect is an exciting concept that can potentially lead to novel methodologies and the production of new materials.

Light-induced olefin metathesis has significantly advanced in the past decade. The excellent spatial control enabled by light, combined with the synthesis of efficient photo-switchable catalysts, has opened many research avenues in the design of chromatic orthogonal and chromatic selective metathesis processes, and especially in polymer and material science applications. The emergence of new generations of photo-switchable catalysts has shifted the activation wavelengths from high-energy UV-light to the visible region, increasing the variety of possible light frequencies. Given the great importance of photoinduced olefin metathesis, several alternative methods to initiate latent catalysts using light have emerged. However, there is still a great need to improve these systems both in terms of reaction scope and efficiency. In this regard, taking advantage of the LSPR effect can provide an extremely efficient process to advance specialized organic reactions such as olefin metathesis.

### BRIEF DESCRIPTION OF THE DRAWINGS

The patent or application file contains at least one drawing executed in color. Copies of this patent or patent application publication with color drawing(s) will be provided by the Office upon request and payment of the necessary fec.

FIG. 1A-1G. present schemes and graphs of Plasmon induced dicyclopentadiene (DCPD) polymerization A. DCPD polymerization reaction scheme and illustrations of different sized gold bipyramids (AuBPs). B. Polymerization temperature profile of DCPD formulation containing AuBP850. Highlighted in grey, an area where an inflection point can be seen in all samples indicating the threshold for the exothermic polymerization reaction. C. Polymerization temperature profile of DCPD formulation containing AuBP660. Grey area as in B. D. E. Image of reaction vials after irradiation. F. “Soft” plasmon induced polymerization temperature profile of AuBP850 containing DCPD. F1. DCPD/cis-Ru—P(OBn)3/AuBP850 formulation. F2.F3. Resulting flexible polydicyclopentadiene (pDCPD). G. “Soft” plasmon induced polymerization temperature profile of AuBP660 containing DCPD. G1. DCPD/cis-Ru—P(OBn)3/AuBP660 formulation. G2.G3. Resulting flexible pDCPD. Thus, by controlling exposure to the activation wavelength the mechanical properties of the polymer can be tuned.

FIG. 2A-2E. present schemes, images and graphs of molding and printing of the plasmonic polymer composites (PPCs). A. Molded PPC rectangles containing different concentrations of AuBPs. Top, PPC660 (embedded with AuBP660, 20 OD on the left and 5 OD on the right) and thermal camera image while irradiated with 660 nm light. Bottom, PPC850 (embedded with AuBP850, 20 OD on the left and 5 OD on the right) and thermal camera image while irradiated with 850 nm light. B. Three molded pDCPD rectangles, on the left PPC660 (20 OD), in the middle containing no AuBPs and on the right PPC850 (20 OD) and thermal camera images while irradiated with 850 nm and 660 nm light respectively. C. Scheme showing the molding process from DCPD/cis-Ru—P(OBn)3/AuBP660 (20 OD) formulation to a fully cured photo-responsive coil. D. Scheme showing the molding process from DCPD/cis-Ru—P(OBn)3/AuBP850 (20 OD) formulation to fully cured photo-responsive knot. E. High-resolution printing of DCPD/cis-Ru—P(OBn)3/AuBP850 (5 OD) formulation and thermal camera image while irradiated with 850 nm light. White scale bar size is 10 mm.

FIG. 3A-3E. present schemes, images and graphs of the photoresponsive reaction vials. A. Scheme showing the preparation and activation of photo-responsive reaction vials that can be utilized to carry out heat activated processes by exposure to light. B. Image of reaction vials coated with PPC850 and PPC660 respectively. C. Temperature profile of 100 heating cycles (30-100° C.) with a PPC850 film (20 OD) by irradiating 850 nm light. D. Zoom-in of the section-colored grey on “C”. E. Images of cycled film before and after completing the heating cycles. The consistency in the duration of the cycles suggests the AuBPs are especially stable when embedded in the polymer.

FIG. 4A-4F. present graph and images of scanning electron microscopy (SEM) presenting the mechanical characterization of AuBP-pDCPD composite. A. DMA E′ onset Tg curves at 10 Hz for the following samples: 1-pDCPD (oven cured), 2-PPC660 (oven cured) 3-PPC660 (plasmon cured), 4-PPC850 (oven cured), 5-PPC850 (plasmon cured). B. Glass transition temperature values of p-DCPD obtained from the Tan δ at 10 Hz. This thermomechanical analysis clearly shows the advantage of plasmon cured PPCs over conventionally cured PPCs. Samples numbered as in 4A. C. SEM image of plasmon cured PPC850. D. SEM image of plasmon cured PPC660. E. SEM image of oven cured PPC850. F. SEM image of oven cured PPC660. The SEM images reveal differences in the structure of AuBP clusters depending on curing method.

### SUMMARY

The following embodiments and aspects thereof are described and illustrated in conjunction with systems, tools and methods which are meant to be exemplary and illustrative, not limiting in scope.

In one aspect of the invention, there is provided a composite comprising (i) a plasmonic material, (ii) a ring-opening metathesis polymerization (ROMP) precursor and (iii) a ROMP catalyst, wherein the ROMP precursor comprises a cycloalkene and an oligomerized derivative of the cycloalkene; and wherein w/w concentration of the oligomerized derivative within the composite is at most 50%; and wherein the plasmonic material is in a form of a plurality of nanoparticles each nanoparticle is encapsulated by an oxide shell; and wherein the plasmonic material is characterized by a photothermal activation wavelength in a range between 400 and 1200 nm.

In one embodiment, a weight ratio between the plasmonic material and the ROMP precursor is between 1:100 and 1:1.000.000.

In one embodiment, a w/w concentration of the plasmonic material within the composite is at least about 0.001%.

In one embodiment, the composite further comprises a latent catalyst.

In one embodiment, the composite is shapeable.

In another aspect, there is provided a composite comprising (i) a plasmonic material (ii) a ROMP catalyst and (iii) a ROM polymer, wherein the plasmonic material is embedded within the polymer; wherein the plasmonic material is in a form of a plurality of nanoparticles each nanoparticle is encapsulated by an oxide shell; wherein the plasmonic material is characterized by a photothermal activation wavelength in a range between 400 and 1200 nm; and wherein a w/w concentration of the plasmonic material within the composite is at least 0.0001%.

In one embodiment, the composite further comprises a latent catalyst.

In one embodiment, the latent catalyst is a catalyst selected from: ring-opening metathesis catalyst, ring-closing metathesis catalyst, ADMET catalyst and olefin metathesis catalyst, including any combination thereof.

In one embodiment, a w/w concentration of the latent catalyst within the composite is at least 0.05%.

In one embodiment, a w/w ratio between the plasmonic material and the ROMP catalyst within the composite is between 1:1 and 1:500.

In one embodiment, plasmonic material is characterized by an average particle size between 100 nm and 500 um.

In one embodiment, plasmonic material comprises plasmonic Au nanoparticles.

In one embodiment, plasmonic Au nanoparticles are gold nano bipyramids (AuBP).

In one embodiment, AuBP is characterized by an average particle size between 10 and 500 nm.

In one embodiment, AuBP is characterized by photothermal activation wavelength between about 600 and about 1200 nm.

In one embodiment, AuBP comprises AuBP660 or AuBP850.

In one embodiment, oxide shell comprises a metalloid oxide, a metal oxide, or both.

In one embodiment, metalloid oxide is silica.

In one embodiment, ROM polymer is in a form of a continuous matrix, and wherein the plasmonic material is embedded within the continuous matrix.

In one embodiment, composite is configured to emit thermal energy upon light irradiation at the photothermal activation wavelength.

In one embodiment, upon a plurality of repetitive photothermal activation and relaxation cycles the composite retains at least 90% of the initial thermal energy.

In one embodiment, plurality of repetitive photothermal activation and relaxation cycles comprises at least 50 cycles.

In one embodiment, thermal energy comprises a temperature increase of the composite by at least 10° C., wherein the increase is measured relative to a temperature of the composite before irradiation.

In one embodiment, continuous matrix comprises a plurality of pores.

In one embodiment, plurality of pores is characterized by an average pore size between 10 and 500 nm.

In one embodiment, composite is a thermoset material; wherein the ROM polymer is polydicyclopentadiene (pDCPD); and wherein the ROMP catalyst is cis-Ru—P(OBn)3.

In another aspect, there is provided an article comprising the composite of the invention such as the polymeric composite of the invention.

In one embodiment, the article is in a form of a film.

In another aspect, there is provided a composite comprising (i) a plasmonic material (ii) a latent catalyst and (iii) polydicyclopentadiene (pDCPD), wherein the plasmonic material is embedded with the pDPCD; wherein the plasmonic material is in a form of a plurality of nanoparticles each nanoparticle is encapsulated by a silica shell; and wherein the plasmonic material is AuBP.

In one embodiment, a w/w concentration of the plasmonic material within the composite is at least 0.001%; and wherein the latent catalyst is selected from: (i) ROMP catalyst, and (ii) olefin metathesis catalyst, including any combination thereof.

In one embodiment, ROMP catalyst is cis-Ru—P(OBn)3.

In one embodiment, olefin metathesis catalyst is selected from cix-Caz-z and cis-Ru—SCF3.

In one embodiment, plasmonic material comprises a plurality of plasmonic Au nanoparticles.

In one embodiment, plasmonic Au nanoparticles are gold nano bipyramids (AuBP).

In one embodiment, AuBP are characterized by an average particle size between 10 and 500 nm.

In one embodiment, AuBPs are characterized by photothermal activation wavelength between about 600 and about 1200 nm.

In one embodiment, AuBP comprises AuBP660 or AuBP850.

In another aspect, there is provided a method of synthesizing the composite the invention, comprising contacting the plasmonic material with a ROMP catalyst and a cycloalkene under appropriate conditions, thereby obtaining a mixture; and polymerizing the mixture by subjecting the mixture to conditions suitable for inducing ROMP of the cycloalkene; wherein the conditions suitable for inducing ROMP comprise: (i) a light irradiation sufficient for inducing a photothermal activation of the plasmonic material; or (ii) conditions sufficient for activation of the ROMP catalyst, thereby, obtaining the composite.

In one embodiment, conditions sufficient for activation of the ROMP catalyst comprise light irradiation at a wavelength range between 200 to 400 nm.

In one embodiment, photothermal activation is sufficient for providing the mixture to a temperature suitable for synthesizing the composite, optionally wherein the temperature is at least about 60° C.

In one embodiment, appropriate conditions comprise contacting for a period time of at least one minute in a solvent and optionally applying ultrasonic waves.

In one embodiment, cycloalkene is capable of undergoing ROMP in the presence of the ROMP catalyst, and wherein the cycloalkane is characterized by a solubility within the solvent of at least 0.1 g/L.

In one embodiment, a w/w ratio between the plasmonic material and the cycloalkene within the mixture is between about 1:1.000.000 and about 1:10; and wherein a w/w ratio between the plasmonic material and the latent catalyst within the mixture is between 1:1 and 1:500.

In one embodiment, cycloalkene is dicyclopentadiene.

In one embodiment, composite is characterized by at least 10% greater glass transition temperature (Tg) as compared to a similar composite manufactured by thermal curing.

In one embodiment, the method further comprises shaping the mixture, thereby obtaining a shaped article comprising the composite.

In one embodiment, shaping is performed prior to the polymerizing.

In one embodiment, shaping is performed by a method selected from casting and molding.

In another aspect, there is provided an article comprising the composite of the invention.

In one embodiment, the article further comprises a latent catalyst.

In one embodiment, the article is a photothermal catalyst.

In one embodiment, the latent catalyst is selected from a ring-opening metathesis catalyst, a ring-closing metathesis catalyst, ADMET catalyst, and an olefin metathesis catalyst, including any combination thereof.

In one embodiment, the article is a mold shaped article.

### DETAILED DESCRIPTION

In some non-limiting aspects of the present invention, there is provided a composite comprising silica encapsulated plasmonic material embedded within a polycycloolefine. In some embodiments, the polycycloolefine is obtained via a ring-opening metathesis polymerization (ROMP). In some embodiments, the plasmonic material is activated by low energy light at visible and near-IR range.

In another aspect, there is provided a method for plasmonic photothermal activation of latent olefin metathesis catalysts using the composite of the invention. The inventors surprisingly observed that due to efficient light-to-heat conversion properties of the plasmonic material it is possible to activate latent olefin metathesis catalyst in a variety of challenging reactions by a contact-free approach using simple LEDs as the light source. The inventors successfully implemented three composites, each composite including a different latent olefin metathesis catalyst as well as two different size gold bipyramids nanoparticles with tunable activation wavelengths, to efficient induce numerous olefin metathesis reactions with a wide scope of possible olefin substrates. Surprisingly, extremely stable photo-responsive dicyclopentadiene-derived polymers embedded with gold bipyramids particles could be produced by either molding or digital light projector printing methodologies, effectively creating efficient heat-emitting low-energy-light responsive composite materials.

### Shapeable Composite

In one aspect of the present invention, there is provided a composite material comprising a ring opening metathesis polymerization (ROMP) precursor, a ROMP catalyst and a plasmonic material, wherein the ROMP precursor comprises a cycloalkene and an oligomerized derivative of the cycloalkene and wherein a w/w concentration of the oligomerized derivative within the composite is at most 50%, and wherein the plasmonic material is in a form of a plurality of nanoparticles, and is characterized by a photothermal activation wavelength in a range between about 400 and about 1200 nm, including any range between; and wherein each nanoparticle is encapsulated by an oxide shell.

As used here, the term “cycloalkene” describes cycloalkene capable of undergoing polymerization so as to form a ROM polymer under conditions suitable for ROM polymerization. In some embodiments, the cycloalkene is an unsaturated mono-, bi- or polycyclic alkane. In some embodiments, the cyclic ring consists of 3, 4, 5, 7 or 8 carbon atoms, and between 1 to 3 carbon-carbon double bond. In some embodiments, the cycloalkene devoid of conjugated dienes and sterically hindered substituents on the double bond. In some embodiments, the cycloalkene may be substituted or unsubstituted by one or more substituents. In some embodiments, the substituent is bound to the cycloalkene at a position which is not the polymerizable double bond. In some embodiments, the substituent is bound to a sp3 carbon of the cycloalkene. In some embodiments, the substituent is devoid of electro-withdrawing groups (e.g., halogen, aldehydes, esters, cyano—etc.).

As used herein the term “oligomerized derivative” refers to a low molecular weight oligomer comprising up to 50 cycloalkene-derived repeating units or to a low molecular weight polymer (e.g., with an average MW of up to 10000 Da). In some embodiments, the oligomerized derivative is linear. In some embodiments, the oligomerized derivative is branched. In some embodiments, the oligomerized derivative is non-crosslinked. In some embodiments, the oligomerized derivative comprises a linear oligomer and a branched (or crosslinked) oligomer, wherein a ratio between the linear oligomer and the branched oligomer is between 1:5 and 5:1, between 1:3 and 1:1, between about 2:1 and 1:2, between about 1:1 and 5:1, or about 1:1, including any range between.

In some embodiments, the oligomerized derivative consists of at most 50, at most 40, at most 30, at most 20, at most 10 repeating units, or between 2 and 50, between 2 and 40, between 2 and 30, between 2 and 20, between 2 and 15, between 2 and 5, between 2 and 10, between 3 and 8, between 8 and 15 repeating units, including any range in between.

A skilled artisan will appreciate that the “cycloalkene-derived repeating unit” refers to a repeating unit of a ring opening metathesis (ROM) polymer (i.e. a polymer obtained via ROMP). As used herein, the terms “ROM polymer” and the term “polyclycloolefin” are used herein interchangeably. The term “ring-opening metathesis polymerization” encompasses a chain-growth polymerization of cyclic alkenes to yield an unsaturated polymer (ROM polymer). In some embodiments, the ROM polymer has an acyclic backbone or a backbone comprising a plurality of cyclic repeating units. In some embodiments, the ROM polymer is a linear polymer or a branched polymer. In some embodiments, the ROM polymer is a crosslinked polymer. In some embodiments, the ROM polymer is a homopolymer or a co-polymer comprising chemically distinct polymeric blocks.

In some embodiments, the ROM polymer of the invention is characterized by an average molecular weight (Mw) between 50.000 and 10.000.000 Da, between 50.000 and 200.000 Da, between 100.000 and 1.000.000 Da, between 100.000 and 500.000 Da, including any range between.

In some embodiments, the ROM polymer of the invention is characterized a PDI of between 1.0 and 1.8, between 1.1 and 1.5, between 1.1 and 1.6, between 1.1 and 1.3 including any range between.

In some embodiments, the ROM polymer of the invention is characterized by Mw and by PDI as disclosed hereinabove.

In one aspect of the present invention, there is provided a shapeable composite material comprising a metathesis polymerization precursor (MPM) consisting essentially of a diene configured to undergo metathesis polymerization, and/or an oligomerized derivative of the diene; a metathesis catalyst (e.g. ROMP or ADMET catalyst); a plasmonic material; and optionally a latent catalyst; wherein a w/w concentration of the MPM within the composite is at least 80%, at least 90%, at least 95%, at least 93%, at least 97%, at least 98%, at least 99%, between 80 and 99.9%, between 80 and 99%, between 80 and 98%, between 80 and 97%, between 80 and 95%, between 90 and 99.9%, between 90 and 99%, between 95 and 99.9%, between 95 and 99%, including any range between; and wherein the plasmonic material is as disclosed herein. In some embodiments, a w/w concentration of (i) the metathesis catalyst, or (ii) the latent catalyst within the shapeable composite is at least at least at least 0.05%, at least 0.1%, at least 0.2%, at least 0.3%, at least 0.5%, at least 1%, and between 0.05 and 5%, between 0.05 and 1%, between 0.05 and 0.5, between 0.1 and 1%, between 0.05 and 0.5%, including any range between.

In some embodiments, a w/w concentration of the ROMP precursor within the shapeable composite material is at least 80%, at least 90%, at least 95%, at least 93%, at least 97%, at least 98%, at least 99%, between 80 and 99.9%, between 80 and 99%, between 80 and 98%, between 80 and 97%, between 80 and 95%, between 90 and 99.9%, between 90 and 99%, between 95 and 99.9%, between 95 and 99%, including any range between.

In some embodiments, a molar ratio between the cycloalkene and the oligomerized derivative within the ROMP precursor is between 10:1 and 1:1, between 10:1 and 2:1, between 8:1 and 2:1, and between 6:1 and 2:1, between 6:1 and 4:1, between 5:1 and 3:1, including any range in between. In some embodiments, a molar ratio between the cycloalkene and the linear oligomerized derivative within the ROMP precursor is about 4:1.

In some embodiments, a w/w ratio between the cycloalkene and the oligomerized derivative within the ROMP precursor is between 10:1 and 1:1, between 10:1 and 2:1, between 8:1 and 2:1, and between 6:1 and 2:1, between 6:1 and 4:1, between 5:1 and 3:1, including any range in between. In some embodiments, a w/w ratio between the cycloalkene and the linear oligomerized derivative within the ROMP precursor is about 4:1.

In some embodiments, a combined weight portion of the linear oligomerized derivative and the cycloalkene within the shapeable composition is between 50 and 99%, between 50 and 90%, between 60 and 99%, between 60 and 90%, between 70 and 99%, between 70 and 90%, between 80 and 99%, between 80 and 90%, including any range in between. In some embodiments, a combined weight portion of the linear oligomerized derivative and the cycloalkene within the shapeable composition is about 80%, or about 82%.

In some embodiments, a weight portion of the branched oligomerized derivative within the shapeable composition is between 1 and 30%, between 1 and 30%, between 1 and 30%, between 1 and 30%, between 1 and 30%, between 1 and 30%, between 1 and 30%, between 1 and 30%. In some embodiments, a combined weight portion of the branched oligomerized derivative within the shapeable composition is about 20%, or about 18%. The weight content of the linear oligomerized derivative and the cycloalkene within the shapeable composite can be determined by determining a weight portion of solid material soluble in an organic solvent (e.g. THF).

In some embodiments, the composite is a shapeable composite. In some embodiments, the shapeable composite encompasses a flexible, elastic, and/or deformable material capable for obtaining a predetermined shape. In some embodiments, the shapeable composite is capable for obtaining a predetermined shape and to further retaining the predetermined shape without applying additional force (i.e., plastic deformation). In some embodiments, the shapeable composite capable for obtaining a predetermined shape at a temperature above glass transition point of the composite. In some embodiments, the shapeable composite capable for obtaining a predetermined shape at a temperature between 0 and 60° C., between 10 and 60° C., between 10 and 50° C., between 10 and 40° C., between 10 and 30° C., including any range between. In some embodiments, the shapeable composite is in a moist state, in a liquid state, or in a semi-liquid state at a temperature between 0 and 50° C., between 0 and 40° C., between 0 and 30° C., between 10 and 30° C., including any range between. In some embodiments, the shapeable composite substantially devoid of phase separation. In some embodiments, the shapeable composite is homogenous. In some embodiments, the shapeable composite is in amorphous state. In some embodiments, the shapeable composite is substantially devoid of crystalline material.

In some embodiments, the shapeable composite is flowable. In some embodiments, the shapeable composite is shapeable by casting or molding (e.g., cast molding, injection molding). In some embodiments, the shapeable composite is characterized by E′ onset determined by DMA, as disclosed herein. In some embodiments, the shapeable composite is characterized by E′ onset of below 50° C., below 40° C., below 35° C., between about 25 and 40° C., between about 25 and 35° C., between about 25 and 33° C., between about 26 and 30° C., including any range between. In some embodiments, E′ onset of the shapeable composite is lower than E′ onset of the polymeric composite by at least 20%, at least 40%, at least 50%, at least 70%, at least 100%, or by at least 10° C., at least 20° C., at least 30° C., at least 50° C., at least 70° C., including any range between.

In some embodiments, the shapeable composite is polymerizable. As used herein the term “polymerizable” refers to the ability of the ROMP precursor to undergo polymerization so as to form a ROM polymer (e.g., a solid thermoplastic polymer, or a solid crosslinked thermoset polymer).

In some embodiments, the cycloalkene is compatible with ROM polymerization. In some embodiments, the cycloalkene is capable of undergoing polymerization so as to form a ROM polymer under conditions suitable for ROM polymerization (e.g., a ROMP catalyst and by providing sufficient activation energy such as by heating the reaction mixture to a suitable temperature).

In some embodiments, the cycloalkene is selected from but not limited to cyclopropene, cyclobutene, cyclopentene, cycloheptene, cyclooctene, norbornene, dicyclopentadiene (DCPD), including any derivative and combination thereof. In some embodiments, the cycloalkene comprises at least one unsaturated 3-5 or 7-9 membered ring. In some embodiments, the cycloalkene is devoid of cyclohexene. As used herein, the term “derivative” refers to a substituted cycloalkene or to a polycyclic cycloalkene (e.g., bi-, or tri-cyclic rings, optionally fused, and further optionally comprising an aliphatic, an aromatic ring, a heteroaromatic ring, a heterocyclic optionally unsaturated aliphatic ring, a cyclic anhydride, etc.). A skilled artisan would appreciate that a suitability of a cycloalkene for ROMP can be assessed based on its strain energy (e.g., a strain energy of a ROMP suitable cycloalkene is above 3, above 5, or above 7 kcal/mol.)

In some embodiments, the cycloalkane is dicyclopentadiene.

In some embodiments, the molar ratio between the dicyclopentadiene and the linear oligomerized derivative (polydiceylopentadiene [pDCPD]) within the precursor is 4:1.

In some embodiments, the shapeable composite further comprises a latent catalyst. The term “latent catalyst” refers to a catalyst that requires activation to induce catalytic activity thereof, wherein activation can occur via heat, light and chemical activation.

In some embodiments, the shapeable composite further comprises an organic solvent (e.g., between 5 and 50% by weight of the composite, including any range between).

In some embodiments, the shapeable composite consists essentially of the ROMP precursor, the ROMP catalyst, the latent catalyst and the plasmonic material. In some embodiments, the shapeable composite consists essentially of the ROMP precursor, the ROMP catalyst, and the plasmonic material.

In some embodiments, between 80 and 100%, between 80 and 99%, between 80 and 95%, between 90 and 100%, between 95 and 99%, or between 95 and 97% of the shapeable composite consists or consist essentially of the ROMP precursor, the ROMP catalyst, the plasmonic material and optionally the latent catalyst.

In some embodiments, the term “composite” and the term “shapeable composite” as used herein, refers to a substantially uniform material which cannot be easily separated into individual constituents (e.g., the particles, and the latent catalyst, ROM polymer). Furthermore, the term “composite” as used herein has distinct physico-chemical properties, as compared to a physical mixture of the constituents of the composite. Uniformity of plasmonic material distribution ca be determined as describe in the example section below.

In some embodiments, the concentration of the oligomerized derivative within shapeable composite is at most 50%, at most 45%, at most 40%, at most 35%, at most 30%, at most 25%, at most 20%, at most 15%, at most 10%, at most 5%, including any range in between. In some embodiments, the concentration of the oligomerized derivative within the shapeable composite is between 1 and 50%, between 5 and 50%, between the 10 and 50%, between 5 and 20%, between 10 and 20%, between 15 and 20%, between 15 and 25%, between 15 and 30%, between 20 and 50%, between 30 and 50%, between 40 and 50%, including any range in between. In some embodiments, the concentration of the oligomerized derivative within the shapeable composite is between 20 and 40%, between 30 and 40%, between 30 and 35%, or about 34%, including any range between.

In some embodiments, a w/w concentration of the plasmonic material within the shapeable composite is at least 0.001%, at least 0.05, at least 0.01, at least 0.05, at least 0.1 at least 0.3%, at least 0.5%, at least 1%, or between 0.001 and 0.1, between 0.01 and 0.1, between, between 0.001 and 0.05, between 0.001 and 1%, between 0.001 and 0.1%, between 0.01 and 0.1%, between 0.01 and 0.5%, including any range between.

In some embodiments, a w/w concentration of (i) the ROMP catalyst, or (ii) the latent catalyst within the shapeable composite is at least at least 0.01, at least 0.05, at least 0.1 at least 0.3%, at least 0.5%, at least 1%, and between 0.01 and 5%, between 0.01 and 1%, between 0.01 and 0.1, between 0.01 and 0.1%, between 0.01 and 0.5%, including any range between.

In some embodiments, the plasmonic material is characterized by a photothermal activation wavelength in a range between about 400 and 1200 nm, between about 400 and 900 nm, between about 400 and 1000 nm, between 500 and 900 nm, between 600 and 900 nm, including any range between.

In some embodiments, the plasmonic material is in a crystalline state. In some embodiments, the plasmonic material is in a semi-crystalline state. In some embodiments, the plasmonic material is in an amorphous state. In some embodiments, the plasmonic material is in a form of metal nanoparticles. In some embodiments, the metal nanoparticles are plasmonic particles. The term “plasmonic particles” is well understood by a skilled artisan and refers to the ability of the particles to convert light to heat via the “localized surface plasmon resonance” (LSPR) effect.

As used herein, the term “substantially” refers to at least 85%, at least 90%, at least 92%, at least 95%, at least 96%, at least 97%, at least 98%, at least 99%, or between 60 and 99.9%, between 70 and 80%, between 70 and 90%, between 80 and 90%, between 90 and 95%, between 95 and 99.9%, including any range or value therebetween.

In some embodiments, the metal nanoparticles are noble metal nanoparticles. In some embodiments, the metal nanoparticles are substantially non-spherical particles. In some embodiments, the metal nanoparticles are enclosed within a support matrix. Without being limited to any particular theory, it is postulated that the support matrix stabilizes the metal nanoparticles, preventing reorganization thereof and thus preventing formation of spherical particles, being substantially devoid of plasmonic properties.

In some embodiments, the plurality of metal nanoparticles is referred to as stable, if at least 80%, at least 85%, at least 90%, at least 95%, at least 99%, by weight of the metal nanoparticles retain at least 90% of the particle size, including any range therebetween.

In some embodiments, the support is an inert matrix encapsulating the plasmonic material (e.g., metal nanoparticles), and is devoid of reactivity towards the plasmonic material. In some embodiments, the support is a porous support. In some embodiments, the support is or comprises an oxide. In some embodiments, the oxide is selected from a metalloid oxide, and a metal oxide (e.g., titanium oxide, zirconium oxide, zinc oxide, aluminum oxide, etc.) In some embodiments, the metalloid oxide comprises silica. In some embodiments, the plasmonic material is in a form of metal nanoparticles embedded within, encapsulated within, or enclosed by an oxide material. In some embodiments, the plasmonic material is in a form of a core shell particle comprising a core consisting essentially of plasmonic metal nanoparticle, and further comprising a shell surrounding the core, wherein the shell consists essentially of porous (e.g., mesoporous) oxide material. In some embodiments, the shell of the core shell particle consists essentially of mesoporous silica. In some embodiments, the support or shell consists essentially of mesoporous silica.

In some embodiments, the plasmonic material comprises or consists essentially of a plasmonic metal particle enclosed within an oxide shell, wherein the plasmonic material is characterized by an average particle size between 0.01 μm and 10 μm, between 1 and 200 nm, between 10 and 200 nm, between 10 and 100 nm, between 50 and 500 nm, between 10 and 500 nm, between 10 and 100 nm, between 100 and 500 nm, between 50 and 300 nm, between 10 and 500 nm, between 50 and 200 nm, between 10 and 200 nm, between 100 and 300 nm, between 300 and 500 nm, between 0.1 μm and 0.5 μm, between 0.5 μm and 10 μm, including any range between. In some embodiments, the average particle size of the plasmonic metal particle refers to a dry particle size (e.g., as determined by SEM).

In some embodiments, the plasmonic material is in a form of noble metal nanoparticles. In some embodiments, the plasmonic material comprises or consist essentially of Au nanoparticles. In some embodiments, the plasmonic material comprises or consist essentially of gold nano bipyramids (AuBP). In some embodiments, the plasmonic material comprises AuBP characterized by photothermal activation wavelength in a range between about 600 and about 1200 nm, between about 600 and about 900 nm, between 700 and 900 nm, between 750 and 900 nm, between 800 and 900 nm, between 600 and 700 nm, including any range between.

In some embodiments, AuBP is AuBP₆₆₀ or AuB₈₅₀, where 660 and 850 refer to their irradiation wavelength respectively.

In some embodiments, a molar ratio between the plasmonic material and the ROMP precursor is between about 1:10 and 1:100.000, between about 1:50 and 1:100.000, between about 1:100 and 1:100.000, between 1:100 and 1:10.000, between 1:100 and 1:5.000, between 1:100 and 1:1.000, including any range in between.

### Polymeric Composite

In another aspect, there is provided a composite (also used herein as “a polymeric composite”) comprising a plasmonic material, a latent catalyst and a polymer, wherein the plasmonic material is embedded within the polymer, and wherein the plasmonic material is in a form of a plurality of nanoparticles each nanoparticle is encapsulated by an oxide shell, and wherein the plasmonic material is characterized by a photothermal activation wavelength in a range between about 400 and about 1200 nm, including any range between; and wherein a w/w concentration of the plasmonic material within the polymeric composite is at least 0.001%. In some embodiments, the polymer is characterized by a melting point (or melting onset) of at least 80, at least 100, at least 150, at least 200° C., or between 80 and 400, between 80 and 300, between 100 and 400, between 100 and 300° C., including any range between. In some embodiments, the polymer is a thermoplastic polymer. In some embodiments, the polymer is a thermoset polymer. In some embodiments, the polymer is an olefin metathesis polymer (e.g. a polymer obtained via metathesis polymerization, such as by ROMP or ADMET). In some embodiments, the polymer is a ROM polymer. In some embodiments, the polymer is a thermoset ROM polymer.

In some embodiments, the polymeric composite is as described hereinabove, wherein the polymer is a thermoset ROM polymer and wherein the latent catalyst is the ROMP catalyst. In some embodiments, the ROM polymer is in amorphous state.

In some embodiments, a w/w concentration of the plasmonic material within the shapeable composite is at least 0.001%, at least 0.05, at least 0.01, at least 0.05, at least 0.1 at least 0.3%, at least 0.5%, at least 1%, or between 0.001 and 0.1, between 0.01 and 0.1, between, between 0.001 and 0.05, between 0.001 and 1%, between 0.001 and 0.1%, between 0.01 and 0.1%, between 0.01 and 0.5%, including any range between.

In some embodiments, the plasmonic material is in amorphous state. In some embodiments, the polymeric composite is substantially devoid of crystalline plasmonic material. In some embodiments, the polymeric composite is in amorphous state.

In some embodiments, the ROM polymer is selected from: polycyclopentene, polycyclobutene, polycycloheptene, polycyclooctene, polydicyclopentadiene, polynorbornene but not limited to, including any derivative, any copolymer or any combination thereof. In some embodiments, ROM polymer is polydicyclopentadiene (pDCPD).

In some embodiments, the ROM polymer is a thermoset polymer.

In some embodiments, the polymeric composite further comprises an additional latent catalyst, which is not metathesis polymerization (e.g., ROMP) catalyst. In some embodiments, the latent catalyst is a catalyst selected from: metathesis polymerization catalyst ring-opening metathesis catalyst, ring-closing metathesis catalyst and olefin metathesis catalyst, but not limited to, including any combination thereof. In some embodiments, the latent catalyst for olefin metathesis is selected from: cis-Caz-z and cis-Ru—SCF₃. In some embodiments, the latent catalyst for ROMP is cis-Ru—P(OBn)3. Additional ROMP catalysts are known in the art such as Titanacyclobutane or tantalacyclobutane complexes, alkylidene catalyst (e.g. Tungsten-carbene complexes, ruthenium-carbene complexes, and ruthenium-carbene complexes bearing NHC ligands).

In some embodiments, the polymeric composite consists essentially of the ROM polymer, the ROMP catalyst, the plasmonic material, and optionally of the latent catalyst.

In some embodiments, between 80 and 100%, between 80 and 99%, between 80 and 95%, between 90 and 100%, between 95 and 99%, or between 95 and 97% of polymeric composite consists, or consist essentially of the ROM polymer, the ROMP catalyst, the plasmonic material, and optionally of the latent catalyst.

In some embodiments, concentration of the latent catalyst within the polymeric composite is at least 0.05%, at least 0.1%, or between 0.05 and 25%, between 0.05 and 0.5%, between 0.1 and 0.5%, between 0.1 and 0.2%, between 0.15 and 0.25%, between 0.1 and 1%, between 0.1 and 5%, between 1 and 5%, between 0.1 and 20%, between 5 and 10%, between 10 and 15%, between 15 and 20%, between 20 and 25%, including any range in between.

In some embodiments, a w/w concentration of (i) the ROMP catalyst, or (ii) the latent catalyst within the polymeric composite is at least at least 0.01, at least 0.05, at least 0.1 at least 0.3%, at least 0.5%, at least 1%, and between 0.01 and 5%, between 0.01 and 1%, between 0.01 and 0.1, between 0.01 and 0.1%, between 0.01 and 0.5%, including any range between.

In some embodiments, a w/w ratio between the plasmonic material and the latent catalyst within the polymeric composite is between 1:1 and 1:500, between 1:1 and 1:50, between 1:1 and 1:10, between 1:1 and 1:15, between 1:1 and 1:20, between 1:10 and 1:30, between 1:10 and 1:100, between 1:10 and 1:500, between 1:5 and 1:500, between 1:5 and 1:300, between 1:5 and 1:200, between 1:50 and 1:500, between 1:10 and 1:30, between 1:20 and 1:40, between 1:20 and 1:30, between 1:30 and 1:40, including any range in between.

In some embodiments, the plasmonic material is as described hereinabove.

In some embodiments, a weight per weight (w/w) ratio between the plasmonic material and the polymer within the polymeric composite is between about 0.001:100 and about 1:10, between 1:10 and 1:1000, between 1:10 and 1:100, between 1:1 and 1:100, between 1:10 and 1:50, between 1:50 and 1:1000, between 1:50 and 1:10,000, between 1:100 and 1:1000, between 1:100 and 1:10,000, between about 1:10 and 1:100.000, between about 1:50 and 1:100.000, between about 1:100 and 1:100.000, between about 1:100 and 1:1.000.000, between about 1:100 and 1:500.000, between 1:100 and 1:10.000, between 1:100 and 1:5.000, between 1:100 and 1:1.000, including any range in between.

In some embodiments, the ROM polymer is in a form of a continuous matrix within the polymeric composite of the invention. In some embodiments, the continuous matrix is porous. In some embodiments, the plasmonic material is embedded within the polymeric matrix. In some embodiments, the plasmonic material is homogenously distributed within the polymeric matrix. In some embodiments, the plurality of particles of the plasmonic material is enclosed by the polymeric matrix. In some embodiments, the particles of the plasmonic material are physisorbed and/or chemisorbed on or within the polymeric matrix. In some embodiments, the plasmonic material is in contact with or bound to the polymer. In some embodiments, the plasmonic particles are substantially located at a pore interphase, wherein the pore interphase is a boundary between the polymer and a void space of the pore.

In some embodiments, the latent catalyst is embedded within the polymeric matrix. In some embodiments, the latent catalyst is physisorbed and/or chemisorbed on or within the polymeric matrix. In some embodiments, the latent catalyst is in contact with or bound to the polymer.

As used herein, the term “matrix” refers to one or more porous layers of polymeric chains that are randomly, and/or under certain order or control, distributed therewithin. Matrix may further include any materials incorporated within and/or interposed between the layers. In some embodiments, the matrix comprises randomly oriented polymeric chains. In some embodiments, each polymeric chain within the matrix is in contact with at least one additional polymeric chain. In some embodiments, the polymeric chains are randomly distributed within the matrix, to obtain a three-dimensional mesh structure comprising a void space between the chains. In some embodiments, the polymeric chains are randomly distributed within the matrix thus forming a plurality of pores (or void space). In some embodiments, the polymeric matrix comprises the thermoset polymer, as described hereinbelow. In some embodiments, the polymeric matrix is an intertwined matrix composed of randomly distributed polymeric chains, the plasmonic material, the latent and ROMP catalyst. In some embodiments, the polymeric chains are randomly distributed within the matrix. In some embodiments, the matrix is substantially devoid of aligned or oriented polymeric chains. In some embodiments, the matrix is substantially devoid of polymeric chains aligned or oriented in a specific direction. In some embodiments, the term “bound” refers to any non-covalent bond or interaction, such as electrostatic bond, dipole-dipole interaction, Van-der-walls' interaction, ionotropic interaction, hydrogen bond, hydrophobic interactions, pi-pi stacking, London forces, etc. In some embodiments, the non-covalent bond or interaction is a stable bond or interaction, wherein stable is as described herein.

In some embodiments, the continuous matrix comprises a plurality of pores. In some embodiments, the plurality of pores is characterized by an average size between 10 and 500 nm, between 10 and 50 nm, between 50 and 100 nm, between 50 and 200 nm, between 50 and 250 nm, between 150 and 250 nm between 100 and 300 nm, between 200 and 300 nm, between 300 and 400, between 400 and 500 nm, including any range in between.

In some embodiments, the term “porosity” as used herein refers to a material comprising pores, holes, voids, or empty space (optionally filled with a gas, such as air or an inert gas), within its network. However, porous layers may optionally comprise an additional substance in the spaces between the molecules of polymer, provided that at least a portion of the volume of the voids is not filled in by the additional substance. In some embodiments, the additional substance comprises the plasmonic material disclosed herein. In some embodiments, the additional substance comprises the latent or ROMP catalyst. Porosity is measured as a fraction (between 0 and 1) of the free volume or pore volume of the porous material relative to the total volume of the porous material, determined by well-known physical measurements, such as N2 adsorption/desorption.

In some embodiment, the porosity of the polymeric composite is between 0.01 and 0.5.

In some embodiment, porosity of the polymeric composite is 0.1, 0.2, 0.3, 0.4, 0.5, between 0.01 and 0.5, between 0.01 and 0.2, between 0.01 and 0.3, including any value and range therebetween.

In some embodiments, the polymeric composite of the invention is referred to as stable, if at least 70% by weight of composition retain at least 75%, at least 80%, at least 85%, at least 90%, and between 75 and 90%, 75 and 85%, 80 and 90%, of their initial energy, including any range therebetween.

In some embodiments, the polymeric composite of the invention is referred to as stable, if at least 70%, at least 80% at least 85%, at least 90%, at least 95%, at least 90% by weight of composition retain at least 90% of their initial energy, including any range therebetween.

In some embodiments, the term “polymeric composite”, as used herein, refers to a substantially uniform material which cannot be easily separated into individual constituents (e.g., the particles, the latent catalyst and the ROM polymer). In some embodiments, a polymeric composite is substantially devoid of phase separation or disintegration (also referred to herein as “stable” polymeric composite). In some embodiments, a polymeric composite is substantially devoid of a multi-layered structure. In some embodiments, the polymeric composite is a homogenous single-layer polymeric composite.

In some embodiments, the term “layer”, refers to a substantially uniform thickness of a material. In some embodiments, the layer or film comprises a single layer, or a plurality of layers. In some embodiments, the term layer and the term film are used herein interchangeably.

In some embodiments, the polymeric composite of the invention is a plasmonic polymeric composite. In some embodiments, the polymeric composite is configured to emit thermal radiation upon light irradiation at the photothermal activation wavelength. In some embodiments, the thermal radiation emitted by the polymeric composite comprises a temperature increase of the polymeric composite by at least 10° C., at least 20° C., at least 30° C., at least 50° C., at least 100° C., at least 150° C., at least 200° C., up to about 500° C., up to about 300° C., up to about 200° C., up to about 150° C., up to about 100° C., including any range between. The temperature increase of the polymeric composite is determined based on a temperature of the same polymeric composite before irradiation. In some embodiments, the polymeric composite upon a plurality of repetitive photothermal activation and relaxation cycles retains at least 90%, at least 85%, at least 80%, at least 75%, at least 70%, and between 70 and 90%, between 70 and 80%, between 75 and 85%, between 80 and 90% of the initial thermal energy. In some embodiments, the plurality of repetitive photothermal activation and relaxation cycles comprises at least 50 cycles and between 50 and 150, between 50 and 80, between 80 and 100, between 90 and 110, between 95 and 120, between 125 and 150 cycles, including any range in between.

In some embodiments, the polymeric composite is a thermoset material.

In another aspect, there is provided a composite, comprising a plasmonic material, a latent catalyst and pDPCD, wherein the plasmonic material is embedded within the pDPCD, and wherein is in a form of a plurality of nanoparticles each nanoparticle is encapsulated by a silica shell, and wherein the plasmonic material is AuBP. In some embodiments, the plasmonic material is characterized by an average particle size between 0.01 μm and 10 μm, between 1 and 200 nm, between 10 and 200 nm, between 10 and 100 nm, between 50 and 500 nm, between 10 and 500 nm, between 10 and 100 nm, between 100 and 500 nm, between 50 and 300 nm, between 10 and 500 nm, between 50 and 200 nm, between 10 and 200 nm, between 100 and 300 nm, between 300 and 500 nm, between 0.1 μm and 0.5 μm, between 0.5 μm and 10 μm, including any range between. In some embodiments, the average particle size of the plasmonic metal particle refers to a dry particle size (e.g., as determined by SEM).

In some embodiments, a w/w concentration of the plasmonic material within the composite is at least 0.001% or between 0.001 and 0.01%; and wherein the latent catalyst is selected from: (i) ROMP catalyst, (ii) ADMET catalyst and (iii) olefin metathesis catalyst, or any combination thereof.

In some embodiments, the ROMP catalyst is cis-Ru—P(OBn)₃.

In some embodiments, the olefin metathesis catalyst is selected from cis-Caz-z and cis-Ru—SCF₃.

In some embodiments, the plasmonic material comprises AuBP characterized by photothermal activation wavelength in a range between about 600 and about 1200 nm, between about 600 and about 900 nm, between 700 and 900 nm, between 750 and 900 nm, between 800 and 900 nm, between 600 and 700 nm, including any range between.

In some embodiments, the AuBP is AuBP660, or AuBP850, or both.

In another aspect, there is provided an article comprising the polymeric composite of the invention. In some embodiments, the polymeric composite further comprises a latent catalyst. In some embodiments, the article is a photothermal catalyst. In some embodiments, the latent catalyst is an olefin metathesis catalyst, capable of undergoing photothermal activation by the plasmonic material disclosed herein. In some embodiments, the latent catalyst is as described hereinabove.

In some embodiments, the photothermal catalyst substantially retains its initial photothermal property upon a plurality of photothermal activation and relaxation cycles. In some embodiments, upon a plurality of repetitive photothermal activation and relaxation cycles, the photothermal catalyst of the invention retains at least 80% at least 85%, at least 90%, at least 95%, at least 97%, at least 99% of the initial photothermal property thereof, wherein the initial photothermal property refers to initial thermal energy emitted by the plasmonic material upon activating thereof (by light irradiation at a suitable wavelength, as disclosed herein). In some embodiments, the plurality of repetitive photothermal activation and relaxation cycles comprises between 2 and 100, between 2 and 50, between 2 and 30, between 10 and 100, between 10 and 50 cycles, including any range between. In some embodiments, each activation and relaxation cycle corresponds to a single catalytic cycle (also referred to herein as “a turnover number”). In some embodiments, the turnover number further encompasses substantial retention of catalytic activity of the latent catalyst.

In some embodiments, photothermal catalyst of the invention is characterized by a turnover number of at least 10, at least 20, at least 30, at east 50, at least 70, at least 90, at least 100, including any range between.

In some embodiments, the article is obtained by shaping and subsequently curing the shapeable composite, as disclosed herein. In some embodiments, the article is a mold shaped article.

In some embodiments, the article (e.g., photothermal catalyst) is in a form of particles (e.g., substantially spherical particles with a particle size between 1 um and 10 mm, including any range between). In some embodiments, the article is in a form of a container (e.g. a reactor) capable of containing a liquid. In some embodiments, the article is in a form of a film. In some embodiments, the film is a transparent film characterized by a transparency between 60 and 100%, including any range between.

In some embodiments, the film is a mono-layer. In some embodiments, the film is a multilayer. In some embodiments, the film layer, is in a form of a continuous layer.

The term “continuous layer” or the term “layer” refers to a substantially homogeneous substance of substantially uniform-thickness which maintains its physico-chemical properties (e.g., glass transition temperature) with the entire dimensions (lengths and width dimensions) thereof. In some embodiments, each layer has a different physical structure and/or a different chemical composition. In some embodiments, each layer has the same physical structure and/or the same chemical composition. In some embodiments, the term “layer”, refers to a polymeric layer.

In some embodiments, the polymeric composite (or the article, such as a film) of the invention is characterized by increased glass transition temperature and/or tensile strength by at least 10%, at least 20%, at least 30%, and between 10 and 75%, between 10 and 30%, between 15 and 40%, between 25 and 35%, between 30 and 50%, between 50 and 75%, including any range in between, compared to a similar oven cured polymeric composite.

In some embodiments, the article is for inducing a reaction catalyzed by the latent catalyst. In some embodiments, the article is for photothermally inducing the reaction. In some embodiments, the reaction is a metathesis reaction (e.g. olefin metathesis; metathesis polymerization, such as ROMP or ADMET; ring-opening metathesis (ROM); ring-closing metathesis (RCM), and olefin metathesis catalyst.

In some embodiments, the article is a photothermal olefin metathesis catalyst. In some embodiments, the article is a photothermal ROMP catalyst. In some embodiments, the article is a photothermal ADMET catalyst. In some embodiments, the article is a photothermal ROM catalyst. In some embodiments, the article is a photothermal RCM catalyst.

### Methods

In another aspect, there is provided a method for synthesizing the polymeric composite of the invention, comprising contacting the plasmonic material disclosed herein with a metathesis polymerization catalyst (e.g. ROMP catalyst or ADMET catalyst) and a monomer suitable for undergoing metathesis polymerization (e.g. a cycloalkene, or a diene) under appropriate conditions, thereby obtaining a mixture; and inducing polymerization of the monomer by subjecting the mixture to conditions suitable for inducing metathesis polymerization of the monomer; wherein the conditions comprise: (i) a light irradiation sufficient for inducing a photothermal activation of the plasmonic material; and/or (ii) conditions sufficient for activation of the metathesis polymerization catalyst, thereby obtaining the polymeric composite. In some embodiments, metathesis polymerization catalyst is a ROMP catalyst or an ADMET catalyst. In some embodiments, the monomer is a diene (suitable for ADMET polymerization) or the cycloalkene described hereinabove.

In another aspect, there is provided a method for synthesizing the polymeric composite of the invention, comprising contacting the plasmonic material disclosed herein with the ROMP catalyst and the cycloalkene under appropriate conditions, thereby obtaining a mixture; and performing polymerization of the cycloalkene by subjecting the mixture to conditions suitable for inducing ROMP of the cycloalkene; wherein the condition suitable for inducing ROMP comprise: (i) a light irradiation sufficient for inducing a photothermal activation of the plasmonic material; and/or (ii) conditions sufficient for activation of the ROMP catalyst, thereby obtaining the polymeric composite.

In some embodiments, polymerization comprises providing the mixture under conditions appropriate for activating the metathesis polymerization catalyst. In some embodiments, conditions appropriate for activating the metathesis polymerization catalyst comprise light irradiating the mixture at a wavelength or at a wavelength range appropriate for activating the plasmonic material, thereby inducing photothermal activation of the metathesis polymerization catalyst. In some embodiments, the wavelength or the wavelength range appropriate for activating the plasmonic material is as described hereinabove (e.g. between about 400 and 1200 nm). In some embodiments, polymerization comprises photothermally activating the metathesis polymerization catalyst, thereby initiating or inducing metathesis polymerization of the monomer.

In some embodiments, photothermal activation comprise light irradiation at a radiation dose sufficient for providing the mixture to a temperature suitable for synthesizing the composite. In some embodiments, temperature suitable for synthesizing the composite is at least about 60° C., at least about 65° C., at least about 70° C., at least about 80° C., at least about 90° C., at least about 100° C., including any range between.

In some embodiments, polymerization comprises light irradiating the mixture at a wavelength or at a wavelength range appropriate for activating the metathesis polymerization catalyst. In some embodiments, the wavelength or the wavelength range appropriate for activating the metathesis polymerization catalyst is a UV radiation (e.g. in a range between 200 to 400 nm, or between about 300 and about 400 nm).

In some embodiments, polymerization comprises light irradiating the mixture at a wavelength at a radiation dose sufficient for activating the plasmonic material, thereby providing the mixture to a temperature suitable for activating the ROMP catalyst to induce ROM polymerization. In some embodiments, the radiation dose is sufficient for providing the mixture (e.g., the liquid composition described hereinbelow) to a temperature at least about 60° C., at least about 70° C., at least about 80° C., at least about 90° C., at least about 100° C., at least about 120° C., at least about 150° C., including any range between.

In some embodiments, polymerization comprises inducing a partial metathesis polymerization of the monomer by light irradiating the mixture at a wavelength or at a wavelength range appropriate for activating the metathesis polymerization catalyst, thereby obtaining the shapeable composite of the invention. In some embodiments, partial polymerization is performed by applying a radiation dose sufficient for inducing polymerization or oligomerization of at least 10% or between 10 and 50% of the monomers.

In some embodiments, the radiation dose is so as to obtain a polymerizable flowable composition, as disclosed herein. In some embodiments, the end point of the partial polymerization is reached when the w/w concentration of the branched metathesis polymer in the mixture is above 30%, above 20%, above 18%, above 15%, above 10%, including any range between.

The concentration of branched metathesis polymer can be determined spectroscopically, or by quantifying the amount of residual material, which is insoluble in an organic solvent, wherein the organic solvent is capable of dissolving the monomer and/or a linear oligomer/polymer. A skilled artisan will appreciate that the specific upper concentration of the branched metathesis polymer in the mixture may vary, depending on the chemical structure of the monomer, and/or viscosity of the mixture. The upper concentration of the branched metathesis polymer at the end of the partial metathesis polymerization step is so as to allow a flow or shaping (i.e. shapeable composite) and subsequent curing of the mixture, as disclosed herein.

Alternatively, the end point of the partial polymerization can be determined by assessing E′ onset by DMA as disclosed in the Examples section. As disclosed hereinabove, the storage modulus (E′) onset of the shapeable polymer (corresponding to the product of the partial polymerization step) is significantly lower than the E′ onset of fully crosslinked thermoset polymer.

In some embodiments, polymerization further comprises a subsequent step (also used herein as “curing”) of exposing the shapeable composite to a light radiation at a wavelength and at a radiation dose sufficient for activating the plasmonic material, as described herein. In some embodiments, curing is performed to obtain a solid thermoset polymer (e.g. the polymeric composite of the invention). In some embodiments, curing is performed by photothermally activating the metathesis polymerization catalyst, thereby polymerizing or curing the shapeable composite so as to obtain a metathesis polymer (e.g. ROMP). A skilled artisan will appreciate that the radiation dose may be varied so as to modify the polymerization (and/or crosslinking degree of the resulting metathesis polymer.

In some embodiments, the method further comprises shaping the shapeable composite, thereby obtaining a shaped article; wherein shaping is performed prior to performing the curing step. In some embodiments, shaping comprises molding or casting. In some embodiments, shaping is for manufacturing an article with a predefined shape.

In some embodiments, the contacting step is performed in a solvent. In some embodiments, the mixture is a liquid composition comprising the monomer (e.g., a cycloalkene, or a diene) and the metathesis polymerization catalyst. In some embodiments, the liquid composition further comprises the plasmonic material dispersed therewithin. In some embodiments, the liquid composition further comprises a latent catalyst dispersed therewithin.

In some embodiments, the contacting step is performed by melting the monomer and adding the plasmonic material, the metathesis polymerization catalyst and optionally the latent catalyst to the molten monomer.

In some embodiments, the mixture comprises a w/w ratio between the plasmonic material and the monomer (e.g. cycloalkene, or diene) within the mixture is between 1:10 and 1:1.000.000, between 1:10 and 1:100, between 1:1 and 1:100, between 1:10 and 1:50, between 1:50 and 1:1000, between 1:50 and 1:10,000, between 1:100 and 1:1000, between 1:100 and 1:10,000, between about 1:10 and 1:100.000, between about 1:50 and 1:100.000, between about 1:100 and 1:1.000.000, between about 1:1000 and 1:100.000, between about 1:1000 and 1:1.000.000, between about 1:100 and 1:500.000, between about 1:100 and 1:100.000, between 1:100 and 1:10.000, between 1:100 and 1:5.000, between 1:100 and 1:1.000, including any range in between. In some embodiments, a concentration of any one of the ROMP catalyst, and the latent catalyst within the mixture is at least 0.05%, at least 0.1%, at least 0.5%, at least 1%, or between 0.1 and 5%, between 0.1 and 2%, between 0.1 and 1%, between 0.05 and 5%, between 0.05 and 1%, between 0.05 and 0.5%, between 0.1 and 5%, between 0.1 and 1%, between 0.1 and 0.5%, including any range between.

In some embodiments, a ratio between the plasmonic material and the latent catalyst within the mixture is between about 1:1 and 1:50, between about 0.1:1 and 1:40 including any range in between.

In some embodiments, contacting comprises a period of time at least one minute (m), at least 0.1 m, at least 3 m, at least 10 m, at least 20 m, at least 30 m, at least 60 m, at least 5 hours, at least 10 hours, including any range between, within the solvent. In some embodiments, appropriate conditions further comprise applying ultrasonic waves.

In some embodiments, the solvent is characterized by a boiling point of at least 35° C., at least 30° C., at least 40° C., at least 50° C., at least 70° C., at least 90° C., including any range between.

In some embodiments, the solvent is suitable for substantially dissolving the monomer. In some embodiments, a solubility of the cycloalkene within the solvent is at least 0.1 g/L, at least 1 g/L, at least 5 g/L, at least 10 g/L, at least 50 g/L, at least 100 g/L, including any range between.

In some embodiments, appropriate conditions comprise a time period of at least one minute (m), at least 0.1 m, at least 3 m, at least 10 m, at least 20 m, at least 30 m, at least 60 m, at least 5 hours, at least 10 hours, including any range between. In some embodiments, appropriate conditions further comprise mixing the liquid composition.

In some embodiments, the subjecting step and the contacting step of the method of the invention are performed simultaneously or subsequently. In some embodiments, the step of synthesizing the composite is performed once. In some embodiments, the step of synthesizing the composite is repeated 1, 2, 3, 4, 5, 6, or more times. In some embodiments, the synthesis step disclosed herein (e.g., the method including the contacting and subjecting steps) is performed in one-pot. In some embodiments, the term “one pot” is meant to refer to a synthesis (or to a process) being carried out in a single reaction vessel, typically, but not exclusively, without removing therefrom any intermediate products.

In some embodiments, the method of the invention is for manufacturing a photothermal catalyst comprising the composite of the invention (e.g. the polymeric composite). In some embodiments, the photothermal catalyst comprises a latent catalyst (e.g. an olefin metathesis catalyst).

In another aspect, there is provided a method for inducing a reaction catalyzed by a latent catalyst, the method comprises contacting an olefin monomer with the photothermal catalyst to obtain a mixture, and exposing the mixture to a light radiation at a wavelength and at a radiation dose sufficient for activating the plasmonic material, thereby inducing the reaction. In some embodiments, exposing is performed by photothermally activating the latent catalyst. In some embodiments, the latent catalyst is an olefin metathesis catalyst, and wherein the reaction is an olefin metathesis reaction.

In some embodiments, the method inducing a reaction disclosed herein is characterized by significantly shorter (e.g. by 2, 10, 100, or by 1000 times) reaction time, and/or by significantly enhanced reaction yield (e.g. by 20%, 50%, 100%, or by 1000% enhanced yield), as compared to a similar reaction devoid of the photothermal catalyst of the invention.

### General

As used herein the terms “about” or “approximately” refer to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

As used herein, the term “substituted” or the term “substituent” are related to one or more (e.g., 2, 3, 4, 5, or 6) substituents, wherein the substituent(s) is as described herein. As used herein, the term substituent comprises halogen, —NO2, —CN, —OH, —CONH2, —CONR′2, —CNNR′2, —CSNR′2, —CONH—OH, —CONH—NH2, —NHCOR, —NHCSR, —NHCNR, —NC(═O)OR, —NC(═O)NR′, —NC(═S)OR′, —NC(═S)NR′, —SO2R′, —SOR′, —SR′, —SO2OR′, —SO2N(R′)2, —NHNR′2, —NNR′, C1-C6 haloalkyl, optionally substituted C1-C6 alkyl, —NH2, —NH(C1-C6 alkyl), —N(C1-C6 alkyl)2, C1-C6 alkoxy, C1-C6 haloalkoxy, hydroxy(C1-C6 alkyl), hydroxy(C1-C6 alkoxy), alkoxy(C1-C6 alkyl), alkoxy(C1-C6 alkoxy), C1-C6 alkyl-NR′2, C1-C6 alkyl-SR′, —CONH(C1-C6 alkyl), —CON(C1-C6 alkyl)2, —CO2H, —CO2R′, —OCOR, —OCOR′, —OC(═O)OR′, —OC(═O)NR′, —OC(═S)OR′, —OC(═S)NR′, or a combination thereof; wherein each R′ independently represents hydrogen, or is selected from the group comprising optionally substituted C1-C10 alkyl, optionally substituted C3-C10 cycloalkyl, optionally substituted C3-C10 heterocyclyl, optionally substituted heteroaryl, optionally substituted aryl, or a combination thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples which, together with the above descriptions, illustrate the invention in a non-limiting fashion.

### Materials

All materials were purchased from Sigma-Aldrich unless noted otherwise. Ultrapure water (type 1, 18.2 MΩ) from Millipore® Direct-Q® 3 with UV was used.

Cetyltrimethylammonium chloride (CTAC) sodium borohydride Reagent Plus 99%, sodium citrate tribasic BioUltra≥99.5%, gold chloride trihydrate 99.9%, cetyltrimethylammonium bromide (CTAB)≥99%, ascorbic acid BioXtra≥99.0%, hydrochloric acid 32%, silver nitrate BioXtra≥99%, tetraethyl orthosilicate (TEOS) reagent grade 98%, ammonium hydroxide 28% in water 99.9%, N,N-Dimethylformamide (DMF) 99.8% for spectroscopy Acros Organics, Zirconium (IV) chloride (ZrCl_{4,}≥99.5%), Terephthalic acid (BDC) 98%, Copper nitrate trihydrate (Cu(NO₃)₂·3H₂O) 99% for analysis ThermoScientific, BTC, Ethanol (EtOH) 99.9% tech Romical, Iron(III) chloride hexahydrate (FeCl₃·6H₂O) ACS reagent 97%, Fumaric acid 99+% Acros Organics, Zinc nitrate hexahydrate (Zn(NO₃)₂·6H₂O) 98% Thermo scientific.

### Methods

### Ultra-Violet Visible (UV-Vis) Light Spectrophotometer

A BioTek Epoch 2 microplate reader equipped with UV-Vis spectrophotometer was used to determine AuBP solution's localized surface plasmon resonance (LSPR) activation wavelength and optical density (OD). Samples were diluted to less than 1 OD in order to obtain accurate measurements and solvents varied depending on experiment requirements.

### Inductively Coupled Plasma Optical Emission Spectrometer (ICP-OES)

The amount of Au was determined using a SPECTRO ARCOS ICP-OES. Samples were prepared by dissolving AuBPs in 2 ml of aqua regia and then diluting to obtain a 10% nitric acid solution.

### Transmission Electron Microscope (TEM)

TEM images were obtained using a Thermo Fisher Scientific (FEI) Talos F200C transmission electron microscope operating at 200 kV. The images were taken with Ceta 16M CMOS camera. Electron Microscopy Sciences formvar/carbon 200 Mesh, copper grids were prepared by adding 5 μl of an AuBP in ethanol solution (2 OD) to the grid surface and allowing it to evaporate under air.

### Scanning Electron Microscope (SEM)

SEM images were obtained using a Thermo Fisher Scientific Verios 460L FEI scanning electron microscope. AuBP embedded polymer samples were prepared by cutting a cross section under liquid nitrogen and carbon coating the sample using an Emitech k575x.

### Dynamic Mechanical Analysis (DMA)

DMA measurements were performed using the Mettler-Toledo Dynamic Mechanical Analyzer (DMA) STARe instrument with a rotatable measuring head. The single cantilever bending approach was used for determining the loss factor tan δ for all polymeric films. Runs with frequencies at 1, 5 and 10 Hz were performed at a constant heating rate of 2° C./min over the temperature ranges from 30° C. to 180° C. A rectangular specimen was used (length: 20 mm; width: 10 mm; thickness: 1 mm). Viscoelastic properties of the cured polymeric films were estimated from the changes in the storage modulus (E′), mechanical loss (E″) as well as from the changes of tan δ (tan δ=E″/E′) at a constant frequency depending on temperature. The Tg was identified as the maximum of the tan δ and the half of the full width from the tan δ curve.

### Tensile Universal Testing Machine (UTM)

Dog-bone-shaped cured specimens of the polymer film were evaluated using a Universal Testing Machine (UTM) (Hounsfield, UK), model H 25 KS, with a maximum load of 25 kN, following the procedure outlined in ASTM D-638 test method. Films were placed between the grips of the testing machine. The grip length was 2.5 cm (the part of the sample held by the instrument), while the loading rate was set to 3 mm/min.

### Matrix-Assisted Laser Desorption/Ionization (MALDI)

Matrix-Assisted Laser Desorption/Ionization (MALDI) mass spectra were recorded on Mass Spectrometer Autoflex speed™ MALDI TOF/TOF. The samples analyzed by MALDI-TOF MS were mixed with NaTFA as additive to improve ion formation.

### Size Exclusion Chromatography (SEC)

Molecular weights and polydispersity indices (PDIs) of the polymers were determined by size exclusion chromatography (SEC) analyses in tetrahydrofuran (THF) at 35□ C using an Agilent 1260 Infinity II Quaternary system HPLC equipped with two Shodex SEC LF-804 Columns and one Shodex SEC KF-803. The flow rate was set to 1 mL/min. Detection was obtained with Agilent 1260 Infinity II Variable Wavelength Detector G7114A, Wyatt ViscoStar WV4-01 viscometer, Wyatt OPTILAB WOP1-01 refractometer, and Wyatt MALS DAWN WD3-02 laser light-scattering system. Prior to measurements, polymer solutions were filtered through Millipore 0.22 μm filters. Wyatt's Astra 8.0.0.25 64-bit software was used for SEC data analysis and polymer properties calculation.

### Nuclear Magnetic Resonance Spectroscopy (NMR)

NMR spectra were obtained with Bruker DPX 400 or DPX 500 instruments; chemical shifts, given in ppm, are relative to the residual solvent peak.

### Gas Chromatography-Mass Spectrometry (GC-MS)

GC-MS data was obtained using an Agilent 6850 GC equipped with an Agilent 5973 MSD working under standard conditions and an Agilent HP5-MS (30*0.25*0.25) column.

Synthesis of AuBPs. Gold bipyramids were synthesized via seed mediated method as reported by (Sánchez-Iglesias, A. et al. High-Yield Seeded Growth of Monodisperse Pentatwinned Gold Nanoparticles through Thermally Induced Seed Twinning. J. Am. Chem. Soc. 139, 107-110 (2017)).

Photothermal synthesis of UIO-66. AuBP@UIO-66 was synthesize via photothermal reaction based on reported solvothermal synthesis (Katz et. al.). Terephthalic acid (123 mg) was dissolved in 10 ml of DMF. ZrCl₄ (123 mg) was dissolved in 10 ml of HCl: DMF mixture (1:5) and aged in moderate temperatures for 30 minutes to enhance dissolving, the two solution were mixed into a vail and different concentrations of AuBPs were added. During the synthesis, the solution was irradiated by LED (850 nm 100 W). The LED operation and temperature monitoring were controlled by Labview program.

Photothermal synthesis of r-MIL-88A. Light induced synthesis of r-MIL-88A was based on a r-MIL-88A solvothermal synthesis reported by (Wang, L. et al. The MIL-88A-Derived Fe 3 O 4-Carbon Hierarchical Nanocomposites for Electrochemical Sensing. Sci. Rep. 5, 1-12 (2015)). 0.4 mmol of FeCl₃·H₂O and 0.4 mmol of fumaric acid were dissolved in 1 ml of ultra-pure water each. Then, the two solutions were mixed and AuBPs were added. The solution was irradiated by 100 W 850 nm LED for 60 minutes at 100° C.

Photothermal synthesis of HKUST-1. Light induced synthesis of HKUST-1 was based on a HKUST-1 microwave assisted synthesis reported by (Seo, Y. K. et al. Microwave synthesis of hybrid inorganic-organic materials including porous Cu3(BTC)2 from Cu(II)-trimesate mixture. Microporous Mesoporous Mater. 119, 331-337 (2009). Chen, B. et al. Synthesis and characterization of the interpenetrated MOF-5. J. Mater. Chem. 20, 3758-3767 (2010)).

0.083 mmol of H₃BTC and 0.152 mmol of Cu(NO₃)₂·3H₂O were dissolved in 1 ml of H₂O:EtOH 1:1 mixture. Then the two solutions were mixed and AuBPs were added. The solution was irradiated by 100W 850 nm LED for 60 minutes at 120° C.

Photothermal synthesis of MOF-5 Light induced synthesis of MOF-5 was based on a synthesis reported by Chen et al. 0.148 mmol of Zn(NO₃)₂·6H₂O and 0.018 mmol of H₂BDC were dissolved in 1 ml of DMF each. Then the two solutions were mixed, 18 μl of ultra-pure water and AuBPs were added. The solution was irradiated by 100 W 850 nm LED for 120 minutes at 120° C.

Photothermal activation of UIO-66. Dried powder of AuBP@UIO-66 putted into a tube, the tube was scaled and connected to air pump. Then the powder was irradiated by 850 nm 100 W LED and the air pump was turned on simultaneously for 10 minutes.

UiO-66@UIO-66 synthesis process. Dried AuBP@UIO-66 powder was dissolved in UIO-66 precursor solution, the solution was then irradiated by 100 W 850 nm LED for 30 minutes at 100° C. The product was washed, dried, and then dissolved in a new UIO-66 precursor solution, to begin a new cycle as described. This was repeated four times including the initial synthesis.

Olefin metathesis catalysts Three different latent olefin metathesis catalysts were used throughout the experiments showing that this method can be utilized to activate a wide scope of thermally active catalysts.

### cis-Caz-1 (Purchased From Strem Chemicals)

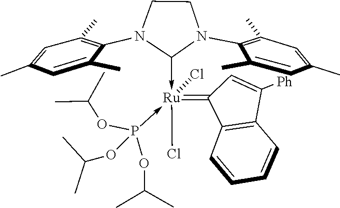 [0209] cis-Ru—P(OBn)₃ (Synthesized as described in Eivgi, O., Vaisman, A., Nechmad, N. B., Baranov, M. and Lemcoff, N. G., Latent Ruthenium Benzylidene Phosphite Complexes for Visible Light Induced Olefin Metathesis, ACS Catal., 2020, 10, 2033-2038).
cis-Ru—SCF₃ (Synthesized as described in Ginzburg, Y., Anaby, A., Vidavsky, Y., Diesendruck, C. E., Ben-Asuly, A., Goldberg, I. and Lemcoff, N. G., Widening the Latency Gap in Chelated Ruthenium Olefin Metathesis Catalysts, Organometallics, 30, 3430-3437, (2011).

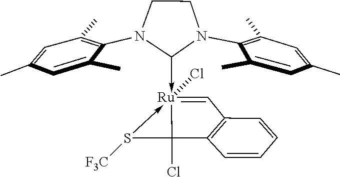

### Temperature Regulation for Plasmon Induced Reactions

The temperature measuring system consists of a MLX90614-DF ROBOT IR Thermometer Sensor connected to a computer via an Arduino Mega. The LEDs used for activating plasmonic AuBPs were purchased from LED ENGIN and connected to a 9130B-BK Precision programmable DC power supply. Data from the IR thermometer and power supply unit was relayed to a Lab View program, enabling the activation of LEDs as a function of the temperature. The systems accuracy was ensured by measuring the temperature of 5 OD AuBPs in 1 ml ethanol with a conventional thermometer (PT 1000.60 Temperature sensor from a RCT 5 digital IKA plate) while utilizing the system to activate the LED and IR thermometer.

### Example 1

### Activation of Olefin Metathesis Latent Catalysts With AuBPs

The efficiency of the plasmonic photothermal process was assessed by activation of commercially available cis-Caz-1. This catalyst is known for its excellent latency at ambient temperatures, exceptional thermal stability, and the ability to complete metathesis reactions of sterically demanding substrates under air when heated. Thus, two challenging ring-closing metathesis (RCM) reactions and the acyclic diene metathesis polymerization (ADMET) of Jojoba oil were explored (Table 1). Notably, just a 5 OD concentration (˜65 ppm) with both types of AuBPs rapidly raised the temperature to 110° C., readily affording full conversions for these difficult RCM reactions when irradiated at their corresponding activation wavelengths: 660 nm light for the 30 nm length AuBPs (AuBP660) and 850 nm wavelength for the 130 nm length AuBPs (AuBP850). In control experiments where reaction mixtures were conventionally heated to the same temperatures, lower conversions were obtained compared to the plasmonic heating (Table 1).

A plausible explanation for this observation could be that the actual temperatures in the near vicinity of the nanoparticles are in reality higher than what is recorded by the thermocouple, thus making the reaction more efficient when this heating method is used. To support this hypothesis, plasmonic photothermal induced reactions at 80° C. were carried out, demonstrating an efficiency comparable to conventional heating at 110° C. This finding may also widen the scope of suitable reaction solvents, enabling the use of lower boiling point solvents that are not normally used due to the disadvantages of performing the reaction at lower reflux temperatures. When considering energy efficiency, the photothermal activation is also favorable. The mixture itself contains the heat source; therefore, only the solution is being heated, in contrast to heating with an oil bath or an oven, where the oil or air are also being heated and demand additional energy. As previously noted, the enhanced reactivity enables the use of lower reaction temperatures or shorter times, further cutting the energy cost of the process.

Additionally, the gold nanoparticles could also be easily recycled from the reaction mixture by simple filtration, and the reaction could be repeated several times without any significant detrimental effect on the overall conversion of the RCM. Remarkably, when conducting the ADMET of jojoba oil, a 10 OD concentration of AuBP660 or AuBP850 raised the reaction temperature to 235° C. or 240° C. respectively when irradiated at the corresponding wavelength, completing the ADMET within an hour and affording a waxy product embedded with AuBPs. Careful size-exclusion chromatography (SEC) analyses of the waxes showed that the reaction promoted by illumination of the nanoparticles produced slightly higher molecular weight oligomers compared to conventional heating (Table 1). Thus, both RCM and ADMET olefin metathesis reactions were shown to be efficiently completed by unprecedented red light or near IR light activation.

TABLE 1

| | | Percent conversion | | | | | |
|---|---|---|---|---|---|---|---|
| Reaction | | | | Cycle number | | | |
| # | RCM (15 min at 110° C.) | | | 1 | 2 | 3 | 4 |
| 1 | 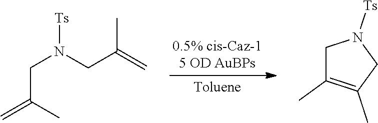 [SMILES: C=C(C)CN(CC(=C)C)S(=O)(=O)c1ccc(C)cc1 ; CC1=C(C)CN(S(=O)(=O)c2ccc(C)cc2)C1] | No AuBP | AuB P₆₆₀ | AuBP₈₅₀ | | | |
| | | 80 | 98 | 97 | 96 | 96 | 94 |
| 2 | 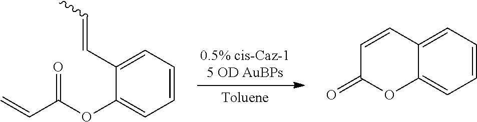 | No AuBP | AuB P₈₅₀ | AuBP₆₆₀ | | | |
| | | 85 | 100 | 100 | 100 | 100 | 100 |

| | | Polymer chain molecular | | |
|---|---|---|---|---|
| | | weight (g/mol) | | |
| | ADMET (1 h at 250° C.) | AuBP₈₅₀ | AuBP₆₆₀ | No AuBP |
| 3 | 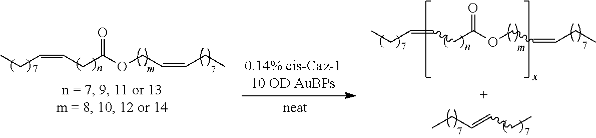 | 3300 | 3300 | 2100 |

### Example 2

### Synthesis of AuBP Embedded Polydicyclopentadiene

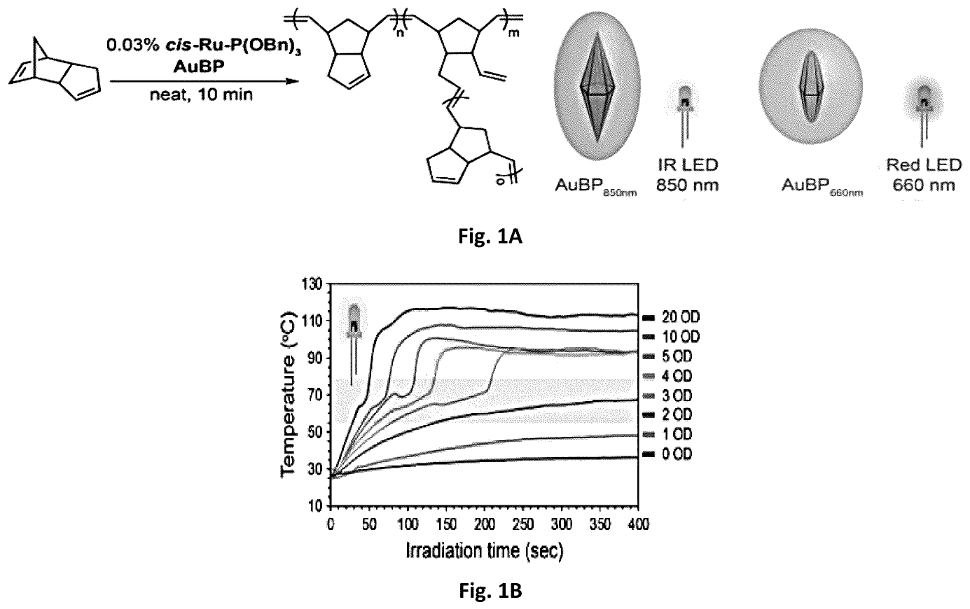

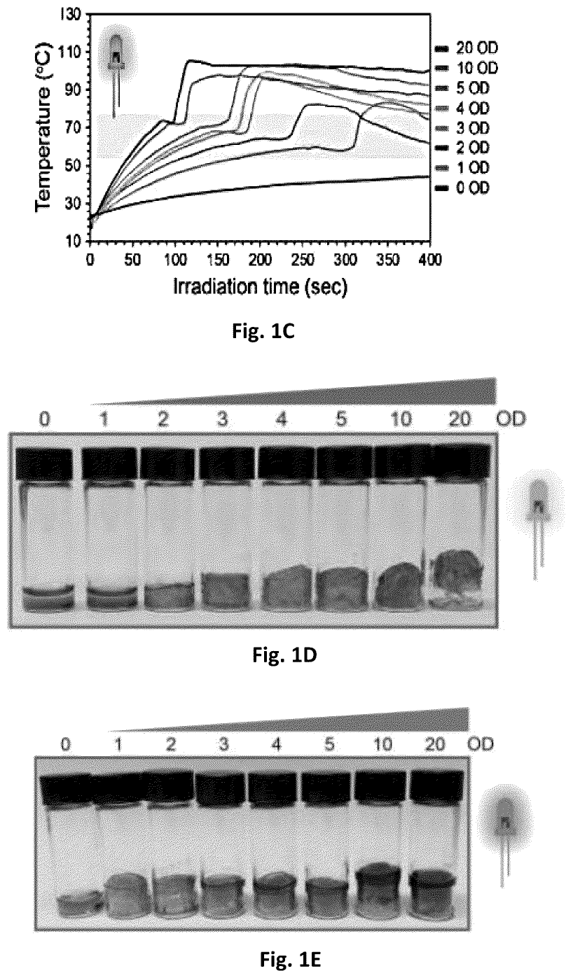

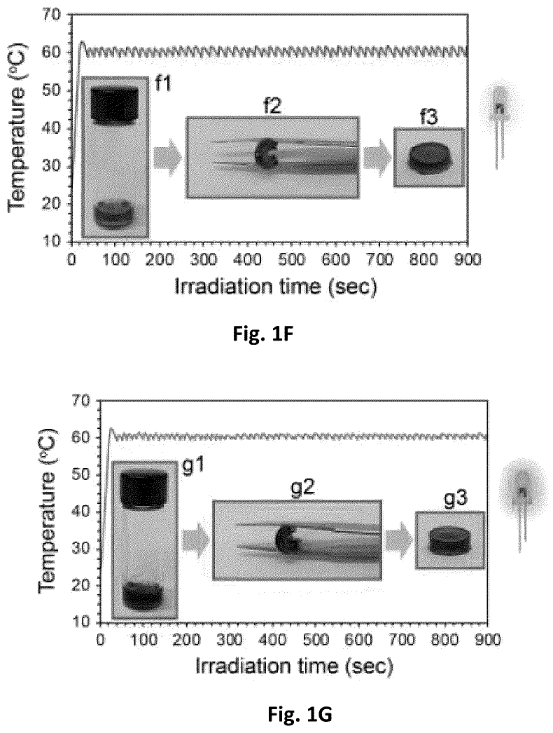

Plasmonic photothermal heating has been used to advance several methodologies in polymerization processes, and plasmonic polymer composites (PPCs) were shown to be effective for several applications. Thus, given their importance in the polymer field, we thought it would be appealing to observe the effect of plasmonic photothermal heating on ROMP reactions and to explore the PPCs that may be obtained by this manner. Dicyclopentadiene (DCPD) derived polymeric materials are known for their excellent thermal and mechanical properties thus, polydicyclopentadiene (pDCPD) is used for applications where mechanical stress, heat and exposure to chemicals and corrosive compounds is typical. However, the industrial utility of these materials is somewhat limited because of the high reactivity of the DCPD monomer. Latent phosphite containing olefin metathesis catalysts may provide a solution to the reactivity problem, as they can produce stable monomer-catalyst formulations with adequate “pot-life”. After demonstrating the capability of AuBPs to carry out RCM and ADMET reactions, we set out to study ROMP efficiency by the preparation of pDCPD materials embedded with AuBPs. In this case, the benzylphosphite catalyst cis-Ru—P(OBn)3 was selected due to its excellent photo-switching properties with UV light and its ability to generate stable and processable formulations with DCPD for up to 2.5 h after mixing. Thus, neat DCPD was mixed with cis-Ru—P(OBn)3 and a specified amount of AuBPs (depending on desired OD). To evaluate the optimal amount of gold bipyramids required for the curing of DCPD, the polymerization's heating profile as a function of AuBP concentration was analyzed (FIG. 1).

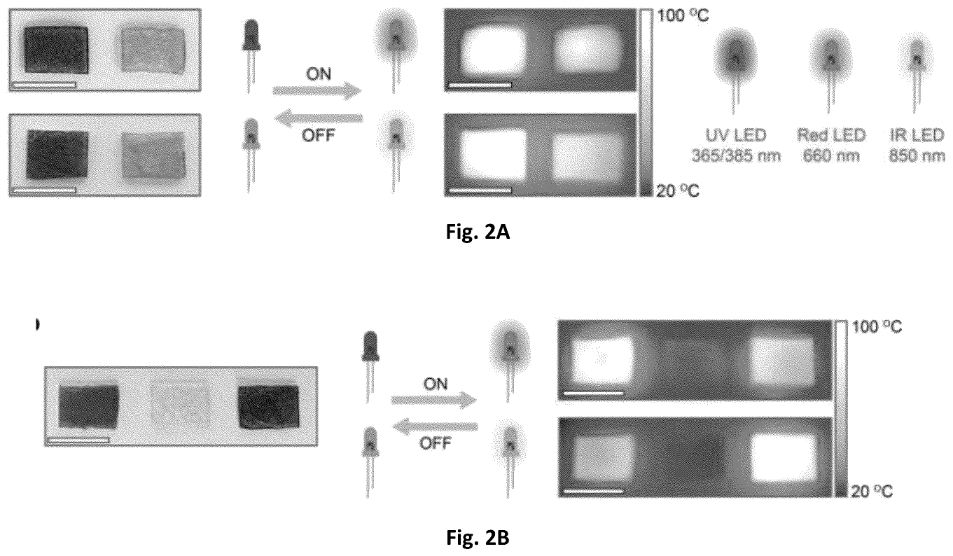

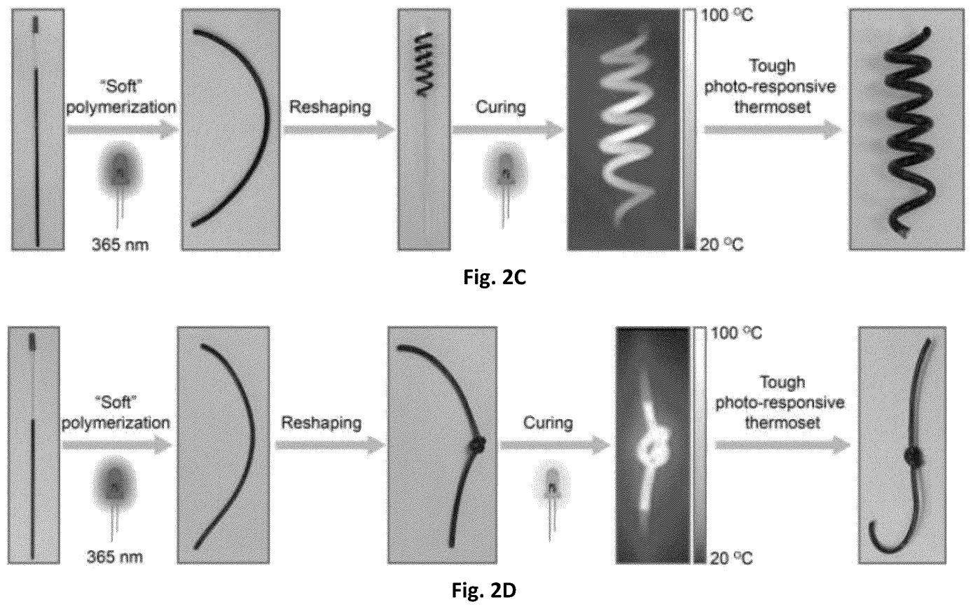

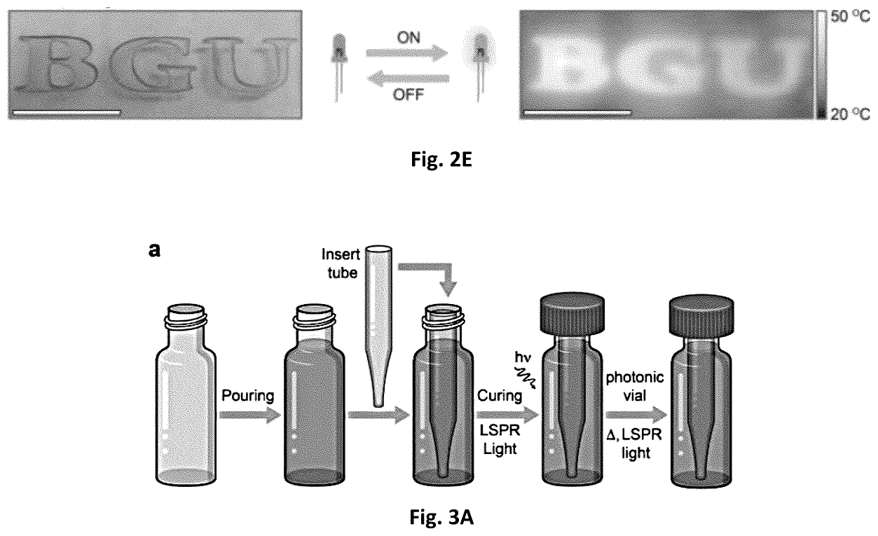

The thermal profiles suggest that efficient curing occurs at a minimum AuBP concentration of 3 OD for IR light (850 nm) and 1 OD for visible light (660 nm), as indicated by the inflection point in the thermal profiles generated by the curing of DCPD. The 660 nm LED generates more heat regardless of the plasmonic effect, as shown in the temperature profiles corresponding to control samples without AuBPs (0 OD), leading to polymerizations at slightly lower AuBP concentrations. Increasing the amount of AuBP850 in the formulation to 20 OD reduces the curing time to 40 seconds while a 20 OD concentration of AuBP660 polymerizes the formulation in 90 seconds. As noted, the plasmonic heating capabilities are highly shape dependent, the sharp tips of the bipyramidal geometry enhance the LSPR absorption bands leading to more efficient photothermal heating. The higher aspect ratios and sharper tips of AuBP850, compared to AuBP660, could be the reason behind the shorter polymerization times observed at 20 OD. Additionally, polymerizing DCPD with AuBPs results in coloring the polymer according to LSPR absorbance (FIG. 1, and FIG. 2).

### Example 3

### AuBP Embedded Plasmonic MOF

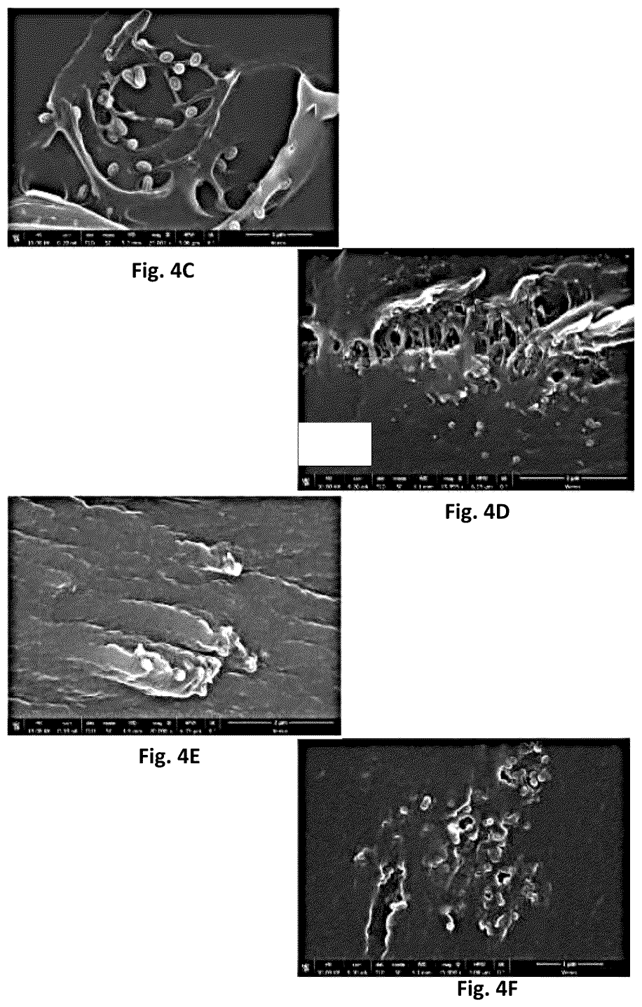

Astonishingly, initial experiments testing the photothermal properties of a washed and dried AuBP₈₅₀ embedded UIO-66 (AuBP₈₅₀@UIO-66), where the composite was irradiated with IR light, produced elevated temperatures surpassing 200° C. (FIG. 4a). Encouraged by the enormous applicative potential of a photo-responsive MOF, Inventors set out to characterize the photothermal abilities of the new material and test it for different applications. To begin with, Inventors prepared three AuBP₈₅₀@UIO-66 samples, as described above, with different concentrations of AuBP₈₅₀ (1, 2 and 5 OD in the initial reaction vial) and irradiated them with 850 nm light while recording the temperature profile (FIG. 4a). Remarkably, the plasmonic UIO-66 synthesized with a 5 OD concentration of AuBP₈₅₀ reached nearly 250° C. in under 5 minutes of exposure. The AuBP₈₅₀@UIO-66 prepared with lower concentrations of nanoparticles also responded to the IR light reaching well over 100° C. To evaluate the stability of the photothermal feature over time Inventors cycled the temperature of a 20 OD-AuBP₈₅₀@UIO-66 between 40-180° C. and 40-100° C. for 3 hours, by turning on and off the NIR LED (FIG. 4b). The results show incredible stability throughout both cycling experiments completing the first and last cycles in approximately the same times. PXRD and BET analyses of the AuBP₈₅₀@UIO-66 after the cycling experiments were performed to ensure the crystalline MOF structure remained intact. Furthermore, very steep heating ramps of 40 to 180° C. in an average of 22 seconds are achieved hinting at the immense applicative potential of the plasmonic photothermal MOF.

Careful inspection of the thermal profiles presented in FIG. 4a revealed an inflection point at around 80° C. recurring in all three curves during the heating process. The Inventors suspected this might be due to the evaporation of leftover solvents trapped in the MOF matrix. Thus, the Inventors envisioned that a photothermal MOF could be highly useful in the photoactivated release of solvents or target molecules adsorbed by the MOF. Another idea the Inventors had in mind from the early stages of the project was to utilize AuBP₈₅₀@UIO-66 as a photothermal agent to induce heat activated reactions, similarly to the way the Inventors used the AuBPs only that now the inherent catalytic properties of the MOF would be functional.

Utilizing MOFs to produce potable water in arid environments by adsorbing water from the air is a promising idea that has shown great applicative potential. Consequently, light induced desorption capabilities of the PMOF were tested by adding increasing amounts of water to a AuBP₈₅₀@UIO-66, irradiating the PMOF with an 850 nm LED, and analyzing the temperature profiles. The inflection point spotted in aforementioned experiments reemerged at roughly the same temperature for all samples, with the exception of a PMOF that was kept dry. Furthermore, increasing the initial volume of water adsorbed by the MOF extended the plateau caused by the evaporation of the adsorbate up to a saturation point where the amount of water the MOF could hold was exceeded (FIG. 5a).

To compare the photothermal desorption mechanism with conventional heating desorption, AuBP₈₅₀@UIO-66 and UIO-66 samples were loaded with identical amounts of water and subjected to both heating methods. For photothermal release the samples were irradiated for one minute to 85° C. (slightly higher than the inflection point observed earlier) with an 850 nm LED, and for conventional heating the samples were kept in an oven set to 95° C. for the same duration. Remarkably, the AuBP850@UIO-66 exposed to NIR released the entire amount of water within a minute, whereas the control samples failed to release even a quarter of the adsorbed water, showcasing the impressive photothermal activity of the plasmonic MOF. To understand the extent of the differences between release techniques a UIO-66 sample was left in the oven until completely dry, ultimately taking 45 minutes to fully desorb the water.

The impressive results obtained for the light induced desorption led us to look for additional ways to exploit the photothermal feature of the new composite. Thus, Inventors decided to try “activating” the MOF via the photothermal response. The porous structure of the MOF can adsorb solvents, moisture and other molecules during synthesis, therefore it is crucial to “activate” the MOF, meaning expelling the adsorbate, before using it. The standard procedure for MOF activation comprises exchanging solvents via repeated centrifugation to a more volatile option and simply placing the MOF in a vacuum oven for prolonged periods of time. To check if the proposed method can efficiently expel the solvent, two identical samples of AuBP₈₅₀@UIO-66 (5 OD) were activated with different methods. A conventionally activated sample was put under vacuum at 120° C. for 17 hours, while the photothermal activation was carried out by exposing the MOF to NIR under vacuum. To ensure the activation of both samples was complete, S_{BET} was examined (FIG. 5). Surprisingly, the photoactivation of the AuBP₈₅₀@UIO-66 was complete after only 5 minutes, an enormous improvement on current conventional procedures.

Finally, the photothermal AuBP₈₅₀@UIO-66 was utilized as a heat source to initiate UIO-66 formation. The plasmonic MOF was added to a UIO-66 precursor solution and the solution was irradiated with NIR light, heating the reaction resulting in UIO-66 synthesis. The product could not be separated from the initial AuBP₈₅₀@UIO-66, essentially affording a photothermal MOF with less AuBPs. This process could be repeated at least 4 times, greatly increasing the amount of MOF that can be synthesized from a given amount of AuBPs. Coupling the MOFs excellent catalytic properties with the ability to create intense heat at its surface can potentially enable very efficient procedures to be carried out by the presented methodology.

Current invention provides the development of a new light induced MOF synthesis utilizing photothermal materials. The novel procedure was found to be robust, versatile, and very rapid, making it an ideal alternative to the conventional time and energy consuming solvothermal syntheses. AuBPs used to generate the heat necessary for UIO-66 formation could be introduced into the MOF in-situ, affording AuBP₈₅₀@UIO-66, demonstrating a new concept for the incorporation of well-defined nanoparticles to MOF matrices. Perhaps the most exciting feature of the MOF-NP composite is the retention of the AuBP's photothermal capabilities, yielding a photoresponsive MOF. Importantly, repeated activation of the photothermal response for up to 3 hours did not affect the efficiency of heat generation or MOF structure, namely S_{BET} and PXRD results remained unaltered. The combination of outstanding light-to-heat conversion with the unique properties of MOFs may serve as a cornerstone for a wide range of potential applications. To highlight the vast impact of what is possible, photothermal desorption, MOF activation and catalysis were performed, affording exciting results. Possibly the most impressive is the completion of MOF activation within a few minutes, while standard procedures can take up to 24 hours. Naturally, Inventors expect to continue investigating the photothermal MOF, testing new opportunities for applications and expanding the insight into the interactions between PNPs and MOFs.

The inventors have successfully manufactured shaped articles using the composites of the invention (e.g. exemplary shapeable composites disclosed herein). Shaping has been performed as follows:

### Molding Technique

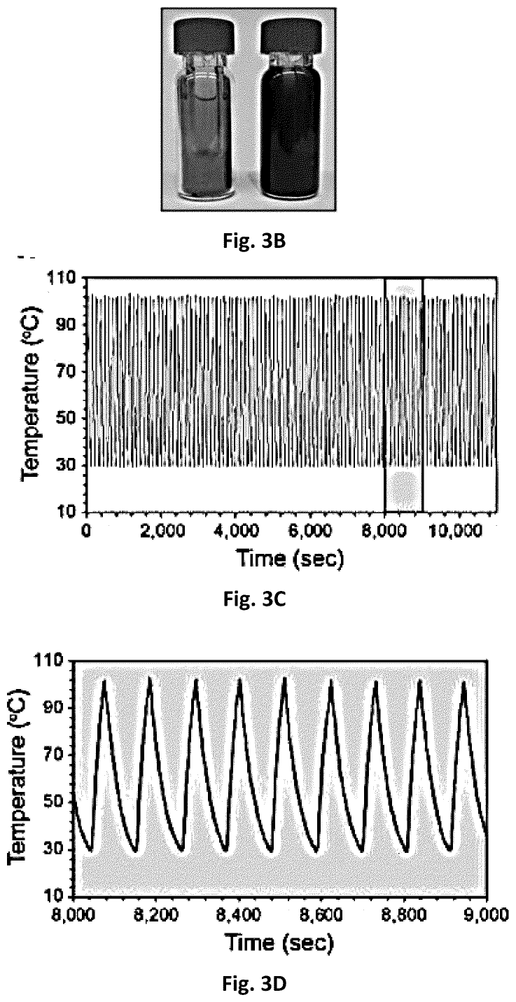

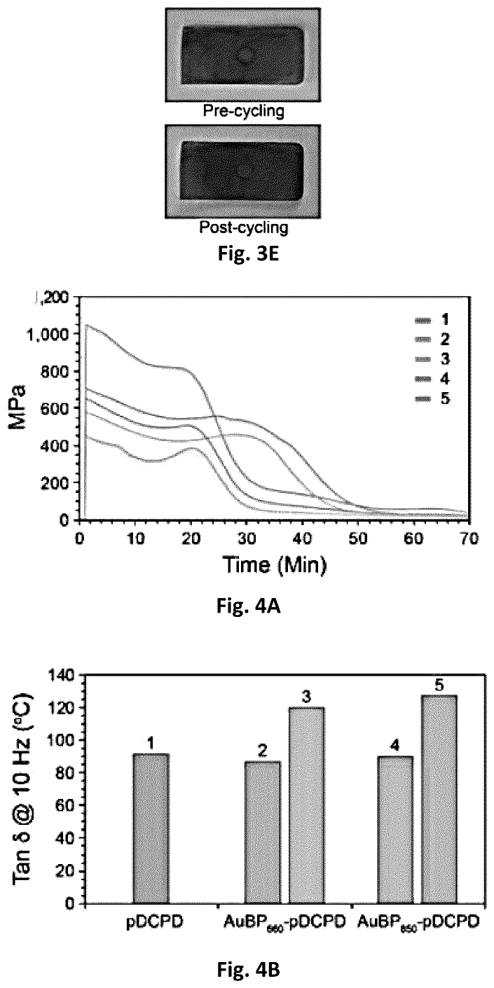

A DCPD/cis-Ru—P(OBn)₃/AuBP formulation was prepared as detailed above. The formulation was inserted into the desired mold (NMR tube or metal mold) and exposed to 365 nm light for 15-45 minutes (for small film molds 15 minutes, large film and dog-bone mold 30 minutes and NMR tubes 45 minutes) until a soft polymer film is obtained. The soft film was then shaped to the desired form (or left in the original mold) and exposed to 850 nm or 660 nm LED (1-5 mins) depending on LSPR activation wavelength. Three types of films were prepared as part of this research using the templates disclosed above. I “Small film” rectangular aluminum template (2.5 cm×1 cm×1 mm). Used for the synthesis of the films seen in FIG. 3 of the article. These films were later cut manually to fit the LED illumination arca. II “Large film” rectangular aluminum template (5 cm×1 cm×1 mm). Used for the synthesis of DMA analysis samples. DCPD/cis-Ru—P(OBn)₃/AuBP₈₅₀ formulation can be seen in the template. III “Dog-bone” aluminum template. Used for the synthesis of UTM analysis samples. The knot and coil presented in FIG. 3 were molded using 3 mm NMR tubes. After “soft” polymerization the tubes were broken carefully and the resulting p-DCPD-AuBP composite was shaped.

Additional ROM polymers has been successfully synthesized based on the exemplary method of the invention.

### ROMP of Cyclooctene

A solution containing 0.5 M cyclooctene, 0.1 mol % cis-Ru—P(OBn)3 and AuBP850 (5 OD) was prepared in toluene. The mixture was then irradiated with an NIR LED (100 watt, 850 nm) for 45 min to 80° C. For the control sample an identical solution was prepared including AuBPs and heated in an oil bath to the same temperature. The yield of photothermal polymerization of cyclooctene was substantially greater, compared to heat-induced polymerization using an oil bath (93% versus 70% conversion).

### Procedure for Solution Uniform Distribution Test

The reaction solution was prepared as described above with AuBP850 and the RCM substrate is

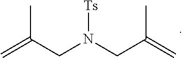 [SMILES: C=C(C)CN(CC(=C)C)S(=O)(=O)c1ccc(C)cc1]

The 0.5 ml solution was then split into three equal volume fractions and their absorbance was measured (diluted 50 times to obtain optimal conditions) to compare the concentration of AuBPs. All three spectra showed the characteristic AuBP850 peak with minor deviations that can be expected when considering slight errors in volume are practically unavoidable, confirming that the original solution was homogenous. Additional validation was obtained by carrying out the reaction separately in each fraction. GC-MS confirmed 99% conversion for all three RCM reactions.

### Procedure for Testing Uniform Distribution in Polymer Composites

A 20 OD PPC850 film was prepared as described above. We then cut the film into three sections and ensured they had an equal thickness so that the absorbance could be compared. The absorbance spectra were then obtained and showed staggering similarity almost completely overlapping. This indicates the AuBPs are very uniformly distributed throughout the PPC.

### Photonic Vials

A DCPD/cis-Ru—P(OBn)3/AuBP850 formulation was poured into a 2 mL dram vial. A glass insert tube was then placed inside the vial, and the formulation was allowed to reach the top of the vial. The DCPD/cis-Ru—P(OBn)3/AuBP850 was fully polymerized by irradiating 850 nm light (100-watt LED) for 5 minutes. The curing of the DCPD-AuBP composite afforded a reaction vessel (the insert) coated by the photo-responsive material, the photonic vial.

0.1 M reaction mixture of diethyl diallyl malonate in toluene was placed inside the photonic vial with a 1 mol % of cis-Ru—SCF3. The vials were exposed to the corresponding activation light (5-watt 850 nm or 660 nm LED) for 15 minutes and reached a temperature of 100° C. Additionally, this reaction was also carried out with AuBP850 directly in the solution as described for previous RCM reactions. The substrate and catalyst concentrations were kept identical, 5 OD of AuBP₈₅₀ were added and the reaction was done at a volume of 0.5 ml.

### Photo-Responsive Characterization

To assess the heating capacity and durability of the photo-responsive feature observed when irradiating the AuBP embedded polymer, a DCPD film containing 850 nm AuBPs at 20 OD was irradiated so that upon reaching 100° C. the LED turned off and when cooling back to 30° C. the LED turned on and reinitiated the plasmonic heating response. The illumination was done with a 100 W 850 nm LED and the temperature was monitored. The inventors observed that the exemplary composite DCPD films are stable upon 100 heating/colling cycles.

### “Soft” Polymer Composition

Initially, a 100 mg UV treated film was stirred in THF for 72 hours. After drying the film, it was weighed and determined that 18% of the original mass was left undissolved. The THF filtrate was analyzed with SEC and showed the presence of a high molecular weight polymer. An identical film was stirred in ethyl acetate and the solvent was analyzed by GC-MS showing the presence of unreacted DCPD. To find the ratio between DCPD monomer and pDCPD, an additional film was prepared, stirred in deuterated THF (72 hours) and analyzed by NMR.

Exemplary soft polymers have been analyzed by DMA, showing storage modulus (E′) onset of between 29 and 31° C. in contrast, fully crosslinked polymers prepared according to the method of the invention were characterized by E′ onset between 110 and 125° C. (see below).

DMA films were initially exposed to 365 nm light for 30 minutes. Then, curing of the films was obtained at 90° C. for 30 minutes by irradiating LSPR activation light (AuBP containing films) or by oven (control films). All films containing AuBPs were prepared with a final concentration of 20 OD. Two identical films (samples 1 and 2) were prepared and tested for each entry.

| | | | Sample 1 | | Sample 2 | |
|---|---|---|---|---|---|---|
| Entry | Formulation | Curing | Onset | Tan δ | Onset | Tan δ |
| 1 | p-DCPD/cis-Ru—P(OBn)₃/No AuBPs | oven | 85° C. | 91° C. | 81° C. | 85° C. |
| 2 | p-DCPD/cis-Ru—P(OBn)₃/AuBP₆₆₀ | oven | 85° C. | 86° C. | 88° C. | 90° C. |
| 3 | p-DCPD/cis-Ru—P(OBn)₃/AuBP₆₆₀ | LED | 113° C. | 119° C. | 114° C. | 121° C. |
| 4 | p-DCPD/cis-Ru—P(OBn)₃/AuBP₈₅₀ | oven | 86° C. | 90° C. | 85° C. | 88° C. |
| 5 | p-DCPD/cis-Ru—P(OBn)₃/AuBP₈₅₀ | LED | 122° C. | 128° C. | 118° C. | 130° C. |

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used. they should not be construed as necessarily limiting.

1. A composite comprising (i) a plasmonic material, (ii) a ring-opening metathesis polymerization (ROMP) precursor and (iii) a ROMP catalyst, wherein said ROMP precursor comprises a cycloalkene and an oligomerized derivative of said cycloalkene; and wherein w/w concentration of said oligomerized derivative within said composite is at most 50%; and
wherein said plasmonic material is in a form of a plurality of nanoparticles each nanoparticle is encapsulated by an oxide shell; and
wherein said plasmonic material is characterized by a photothermal activation wavelength in a range between 400 and 1200 nm.

2. The composite of claim 1, wherein a weight ratio between said plasmonic material and said ROMP precursor is between 1:100 and 1:1.000.000.

3. The composite of claim 1, wherein a w/w concentration of said plasmonic material within said composite is at least about 0.001%.

4. The composition of claim 1, wherein said composite is selected from (a) a composite further comprising a latent catalyst, (b) a shapeable composite, and (c) both (a) and (b).

5. (canceled)

6. A composite comprising (i) a plasmonic material (ii) a ROMP catalyst and (iii) a ROM polymer, wherein said plasmonic material is embedded within said polymer; and
wherein said plasmonic material is in a form of a plurality of nanoparticles each nanoparticle is encapsulated by an oxide shell; and
wherein said plasmonic material is characterized by a photothermal activation wavelength in a range between 400 and 1200 nm; and
wherein a w/w concentration of said plasmonic material within said composite is at least 0.0001%.

7. The composite of claim 6, wherein said composite further comprises a latent catalyst.

8. The composite of claim 7, wherein any one of: (a) said latent catalyst is a catalyst selected from: ring-opening metathesis catalyst, ring-closing metathesis catalyst, ADMET catalyst and olefin metathesis catalyst, including any combination thereof; (b) a w/w concentration of said latent catalyst within said composite is at least 0.05%; (c) a w/w ratio between said plasmonic material and said ROMP catalyst within said composite is between 1:1 and 1:500; and (d) said plasmonic material is characterized by an average particle size between 100 nm and 500 um.

9.-11. (canceled)

12. The composite of claim 6, wherein said plasmonic material comprises plasmonic Au nanoparticles optionally wherein said plasmonic Au nanoparticles are gold nano bipyramids (AuBP), further optionally wherein said AuBP is characterized by any one of an average particle size between 10 and 500 nm, photothermal activation wavelength between about 600 and about 1200 nm, said AuBP comprises AuBP660 or AuBP850.

13.-16. (canceled)

17. The composite of claim 6 wherein said oxide shell comprises a metalloid oxide, a metal oxide, or both, optionally wherein said metalloid oxide is silica.

18. (canceled)

19. The composite of claim 6, wherein any one of: (a) said ROM polymer is in a form of a continuous matrix, and wherein said plasmonic material is embedded within said continuous matrix; (b) said composite is configured to emit thermal energy upon light irradiation at the photothermal activation wavelength; (c) upon a plurality of repetitive photothermal activation and relaxation cycles said composite retains at least 90% of the initial thermal energy, optionally wherein said plurality of repetitive photothermal activation and relaxation cycles comprises at least 50 cycles; (d) said composite is a thermoset material; wherein said ROM polymer is polydicyclopentadiene (pDCPD); and wherein said ROMP catalyst is cis-Ru—P(OBn)3.

20.-23. (canceled)

24. The composite of claim 19, wherein said continuous matrix comprises a plurality of pores, optionally wherein said plurality of pores is characterized by an average pore size between 10 and 500 nm.

25.-26. (canceled)

27. An article comprising the composite of claim 1, optionally wherein said article is in a form of a film.

28. (canceled)

29. A composite comprising (i) a plasmonic material (ii) a latent catalyst and (iii) polydicyclopentadiene (pDCPD), wherein said plasmonic material is embedded with said pDPCD;
wherein said plasmonic material is in a form of a plurality of nanoparticles each nanoparticle is encapsulated by a silica shell; and wherein said plasmonic material is AuBP.

30. The composite of claim 29, wherein a w/w concentration of said plasmonic material within said composite is at least 0.001%; and wherein said latent catalyst is selected from: (i) ROMP catalyst, and (ii) olefin metathesis catalyst, including any combination thereof.

31. The composite of claim 30, wherein said ROMP catalyst is cis-Ru—P(OBn)₃.

32. The composite of claim 29, wherein said olefin metathesis catalyst is selected from cix-Caz-z and cis-Ru—SCF₃.

33. The composite of any of claim 29, wherein said plasmonic material is selected from plasmonic material comprising a plurality of plasmonic Au nanoparticles, and plasmonic Au nanoparticles are gold nano bipyramids (AuBP).

34. (canceled)

35. The composite of claim 33, wherein said AuBP are characterized by (a) an average particle size between 10 and 500 nm; (b) photothermal activation wavelength between about 600 and about 1200 nm; (c) AuBP compring AuBP660 or AuBP850.

36.-37. (canceled)

38. A method of synthesizing the composite of claim 6, comprising contacting said plasmonic material with a ROMP catalyst and a cycloalkene under appropriate conditions, thereby obtaining a mixture; and polymerizing said mixture by subjecting said mixture to conditions suitable for inducing ROMP of said cycloalkene; wherein said conditions suitable for inducing ROMP comprise: (i) a light irradiation sufficient for inducing a photothermal activation of said plasmonic material; or (ii) conditions sufficient for activation of said ROMP catalyst, thereby, obtaining said composite.

39. The method of claim 38, wherein any one of (a) said conditions sufficient for activation of said ROMP catalyst comprise light irradiation at a wavelength range between 200 to 400 nm; (b) said photothermal activation is sufficient for providing said mixture to a temperature suitable for synthesizing said composite, optionally wherein said temperature is at least about 60° C.; (c) said appropriate conditions comprise contacting for a period time of at least one minute in a solvent and optionally applying ultrasonic waves; (d) said cycloalkene is capable of undergoing ROMP in the presence of said ROMP catalyst, and wherein said cycloalkane is characterized by a solubility within said solvent of at least 0.1 g/L; (e) a w/w ratio between said plasmonic material and said cycloalkene within said mixture is between about 1:1.000.000 and about 1:10; and wherein a w/w ratio between said plasmonic material and said latent catalyst within said mixture is between 1:1 and 1:500; (f) said cycloalkene is dicyclopentadiene; (g) said composite is characterized by at least 10% greater glass transition temperature (Tg) as compared to a similar composite manufactured by thermal curing.

40.-53. (canceled)
